(19) United States
(12) Patent Application Publication
(10) Pub. No.: US 2018/0135010 A1
(43) Pub. Date: May 17, 2018

(54) ISOLATION AND LONG-TERM CULTURING OF ESTROGEN RECEPTOR-POSITIVE HUMAN BREAST EPITHELIAL CELLS

(71) Applicant: University of Copenhagen, Copenhagen K (DK)

(72) Inventors: Agla Jael Rubner Fridriksdottir, Copenhagen SV (DK); Jiyoung Kim, Valby (DK); René Villadsen, Frederikssund (DK); Branden Hopkinsson, Copenhagen N (DK); Marie Christine Klitgaard, Copenhagen Ø (DK); Ole William Petersen, Værløse (DK); Lone Rønnov-Jessen Petersen, Værløse (DK)

(21) Appl. No.: 15/573,349

(22) PCT Filed: May 11, 2016
(86) PCT No.: PCT/DK2016/050122

§ 371 Date: Nov. 10, 2017

(30) Foreign Application Priority Data
May 11, 2015 (DK) PA 2015 70274

(51) Int. Cl. C12N 5/071; G01N 33/50

(52) U.S. Cl. CPC C12N 5/0631; G01N 33/5091; C12N 2500/25; C12N 2500/46; C12N 2501/11; C12N 2510/04; C12N 2501/15; C12N 2501/392; C12N 2501/727; C12N 2501/998; C12N 2503/02; C12N 2501/115

(57) ABSTRACT

The present invention describes methods for long-term culturing of ER^{pos} cells, cell lines, and/or cell strains with exetended lifespan and/or cell strain as well as culture medium compositions. The invention further describes methods for isolating cells which may be used as starting point for long-term culturing of ER^{pos} cells, cell lines, and/or cell strains with extended lifespan and/or cell strain. The invention further discloses various methods for generating ER^{pos} tumorigenic cells, cell lines, and/or cell strains with extended lifespan and/or cell strain as well as various assays for their use.

### FIELD OF INVENTION

The present invention relates to methods and culture media for long-term culturing of estrogen receptor positive (ER^{pos}) cells such as primary cultures, cell strains with extended lifespan and immortalized cell lines with an ER^{pos} phenotype. The present invention further relates to methods for isolating cells capable of yielding long-term ER^{pos} cell cultures. The present invention is clinically relevant because it may help explain an enigmatic difference between the normal human breast and breast cancers, a prerequisite for developing new therapies against breast cancer.

### BACKGROUND OF INVENTION

Understanding the taxonomy and evolution of breast cancer has always relied heavily on the use of normal cell types as reference. Nevertheless, ever since the first protocol for cultivation of normal human breast epithelial cells appeared three decades ago, it has become increasingly clear that there are no protocols that support propagation of ER^{pos} cells or even maintenance beyond a few days in culture. Thus, along with the appreciation of epithelial cell lineages in the human breast, primarily the luminal lineage and the basal/myoepithelial lineage, it became evident that the fastest growing cells in culture are of basal origin. Moreover, when it was revealed that ER^{pos} cells in situ accounted for only by average seven percent (mean 6.6%, ranging from 1.2-19.1% in a series of 15 normal breast samples) of the cells within the luminal epithelial lineage, the chances of recovering these cells in culture without prospective isolation would in many cases be elusive. Thus, in culture medium that allowed luminal cells to be maintained in culture after passaging, endogenous ER expression disappeared. Likewise, even when employing freshly isolated small pieces of breast tissue, including the surrounding stroma thus preserving tissue architecture, steroid receptor expression is eventually lost after few days. As a consequence of this, the comparison of cancer with “normal”, for example the HMT-3522, MCF10A, and 184B5 cell lines, in cell based assays has relied on normal cells lacking ER expression.

In an attempt to overcome the loss of receptor expression, ER has been ectopically introduced into such cell lines. This approach, however, has had a number of shortcomings, e. g. instead of responding to estrogen by increased proliferation as expected, the ER-transfected cells show growth inhibition. Accordingly, most of our current knowledge of ER expression, regulation and action comes from breast carcinoma cell lines, where their relation to ER^{pos} normal breast cells at best remains speculative.

Investigating the susceptibility of estrogen receptor positive (ER^{pos}) normal human breast epithelial cells (HBEC) for clinical purposes or basic research awaits a proficient cell based assay.

The prospective isolation and tracking of ER^{pos} single cells from normal breast hold promises for the future comparisons between normal, benign and malignant ER^{pos} cells, which will hopefully shed some light on the evolution and pathogenesis of the most frequent form of human breast cancer. Being able to isolate and track the cells, however, would be of limited value if the ER expression was lost upon culture. It has been anticipated that ER^{pos} normal cells cannot divide and that this is why human breast epithelial cells rapidly lose ER expression in culture.

In spite of the fact that there are multiple protocols for enrichment, long-term cultivation and clonal growth of human breast epithelial cells, none of them are able to isolate, track or support growth of ER^{pos} human breast epithelial cells.

Consequently there is a need to develop culture methods that allow cells to be maintained in culture while maintaining endogenous ER expression.

### SUMMARY OF INVENTION

The present invention is the result of several years of experimentation including many reduction mammoplasties from different donors until the surprising finding that the conditions disclosed herein permit the isolation and culturing of ER positive cells. The present inventors have identified markers for isolating ER^{pos} cells and to coax what appear to be post-mitotic primary cells into exponentially growing long-term ER^{pos} cell cultures and cell strains with extended lifespan. The present inventors report a robust technique for isolating and tracking ER^{pos} human breast epithelial cells from reduction mammoplasties by FACS using cell surface markers including CD166 and CD117, and/or an intracellular cytokeratin marker, Ks20.8, for further tracking single cells in culture. In addition the present inventors show that ER^{pos} human breast epithelial cells are released from growth restraint by small molecule inhibitors of TGF-β signaling. The present inventors further herein demonstrate estrogen responsiveness after numerous population doublings, thereby showing long term culturing of ER^{pos} cells. Importantly, ER signaling is functionally active also in ER^{pos} cells in long-term culture.

The findings of the present invention open an entirely new avenue of experimentation with normal ER^{pos} human breast epithelial cells and provide a basis for understanding the evolution of human breast cancer, which is a prerequisite for developing new therapies against the most frequent form of human breast cancer.

Thus a major aspect of the present invention relates to an immortalized estrogen receptor positive cell line, wherein said cell line has a CD326^{high}/CD271^{low} origin.

Another aspect of the present invention relates to a method of isolating a primary breast epithelial cell which is capable of establishing the estrogen receptor positive cell line described herein and/or the estrogen receptor positive cell strain with extended lifespan as described herein, said method comprising the steps of: a) providing a sample of breast epithelium cells; and b) isolating a primary cell with a CD326^{high}/CD271^{low} phenotype, thereby isolating a primary breast epithelial cell capable of yielding a cell line as described herein and/or a cell strain with extended lifespan as described herein.

An important aspect of the present invention relates to a method of generating an estrogen receptor positive cell strain with extended lifespan from an isolated breast epithelial cell, the method comprising the steps of: a) isolating a breast epithelial cell as described herein; and b) culturing said isolated cell in presence of at least one feeder cell in culture medium B as described herein, c) isolating a cell with a CD326^{high}/CD271^{high} or a CD326^{high}/CD271^{low} phenotype, and d) culturing said isolated cell of c in culture medium A as described herein, wherein said isolated cell generates an estrogen receptor positive cell strain with extended lifespan capable of responding to estrogen.

The inventors have further shown that Ks20.8 (originally raised against cytokeratin 20) is a novel ER^{pos} cell marker. Thus another aspect of the invention relates to a method of generating an estrogen receptor positive cell strain from an isolated breast epithelial cell, the method comprising the steps of: a) isolating a breast epithelial cell as described herein; and b) culturing said isolated cell in culture medium A as described herein, wherein said isolated cell generates an estrogen receptor positive cell strain capable of responding to estrogen.

The inventors have further shown that Ks20.8 (originally raised against cytokeratin 20) is a novel intracellular marker for ER^{pos} cell. Thus another aspect of the present invention relates to a method of distinguishing an estrogen receptor positive cell and an estrogen receptor negative cell based on cell surface proteins as described herein, said method comprising the steps of: a) providing a cell, b) determining if said cell has a Ks20.8^{high} or Ks20.8^{low} phenotype, wherein a cell with a Ks.20.8^{high} phenotype is estrogen receptor positive and a cell with a Ks20.8^{low} phenotype is estrogen receptor negative. The antibody (clone Ks20.8) does not bind cytokeratin 20 (CK20) in breast cells since CK20 is not expressed in such cells. Instead the inventors suggest that Ks20.8 cross-reacts with cytokeratin 8 (CK8). However, the inventors have shown that Ks20.8 can be utilized for identifying CD166^{high} cells independent of ER expression. However, ER positive cells may also be obtained from CD166^{low}/CD117^{high} cells.

A further aspect of the present invention relates to a cell culture medium A for inducing and/or maintaining an estrogen receptor positive phenotype in a primary breast epithelial cell, cell strain with extended lifespan and/or a cell line as described by the present invention, wherein the culture medium comprises an inhibitor of a TGF-β type I receptor.

A further aspect of the present invention relates to a culture medium B for generating a CD326^{high}/CD271^{high} breast epithelial cell, wherein the culture medium comprises a Rho-associated coiled coil forming protein serine/threonine kinase inhibitor, adenine and/or a serum replacement, such as B27.

Yet another aspect of the present invention relates to a method for producing a tumorigenic cell from an estrogen receptor positive cell, cell strain with extended lifespan and/or cell line, the method comprising the steps of: a) providing estrogen receptor positive cell, cell strain with extended lifespan and/or cell line and/or cell strain as described by the present invention, and b) contacting said cell to a tumorigenic agent which transforms said cell, cell strain with extended lifespan and/or cell line into tumorigenic cells capable of forming a tumor in vivo and/or exhibiting tumor cell characteristics in culture.

Another aspect of the present invention relates to a tumorigenic cell, cell strain with extended lifespan and/or cell line and/or cell strain produced as described by the present invention.

A further aspect of the present invention relates to a culture method for identifying an agent which reduces proliferation and/or survival of a tumorigenic cell, cell strain with extended lifespan and/or cell line and/or cell strain described by the present invention, comprising the steps of: a) contacting a tumorigenic cell, cell strain with extended lifespan and/or cell line described by the present invention with a candidate agent; b) assessing the ability of said candidate agent to reduce proliferation and/or survival of said tumorigenic cell, cell strain with extended lifespan and/or cell line and/or cell strain, c) determining the extent to which proliferation of the tumorigenic cell, cell strain with extended lifespan and/or cell line and/or cell strain occurs in the presence of the candidate agent; and d) comparing the extent determined with the extent to which proliferation of the tumorigenic cell, cell strain with extended lifespan and/or cell line and/or cell strain occurs under the same conditions, but in absence of the candidate agent, wherein if the proliferation and/or survival occurs to a lesser extent in the presence of the candidate agent than in its absence, the candidate agent is an agent which reduces proliferation and/or survival of said tumorigenic cell, cell strain with extended lifespan and/or cell line and/or cell strain.

Yet an aspect of the present invention relates to a method of identifying a gene the expression of which in a tumorigenic cell, cell strain with extended lifespan and/or cell line and/or cell strain as described by the present invention is related to or involved in metastasis of such cell in vivo, the method comprising the steps of: a) introducing a candidate gene into a tumorigenic cell, cell strain with extended lifespan and/or cell line as described by the present invention thereby producing a modified tumorigenic cell, cell strain with extended lifespan and/or cell line; b) introducing the modified tumorigenic cell, cell strain with extended lifespan and/or cell line into a test animal; c) maintaining said test animal under conditions appropriate for metastasis to occur; and d) determining whether metastasis of the modified tumorigenic cell, cell strain with extended lifespan and/or cell line occur, wherein if metastasis occurs, the candidate gene is a gene the expression of which in a tumorigenic cell, cell strain with extended lifespan and/or cell line and/or cell strain is related to or involved in metastasis of such cells in vivo.

Another aspect of the present invention relates to a method of identifying a gene product which is upregulated or downregulated in a tumor cell, compared to a normal cell of the same type, the method comprising the steps of: a) analyzing the tumorigenic cell, cell strain with extended lifespan and/or cell line and/or cell strain described by the present invention; b) analysing a normal cell from which said tumorigenic cell, cell strain with extended lifespan and/or cell line and/or cell strain is derived from; and c) comparing the gene products produced in step (a) with step (b), whereby a gene product which is upregulated or downregulated is identified.

A further aspect of the present invention relates to a method of culturing cancer cells, tumorigenic cells and/or tumors from normal and/or luminal epithelial cells, wherein said normal and/or luminal epithelial cells are grown in a cholera toxin free medium comprising FAD2, TGFβR inhibitor, with or without estrogen or in BBMYAB with or without estrogen on fibroblast feeders as described by the present invention.

A further aspect of the present invention relates to a method of culturing cancer cells, tumorigenic cells and/or tumors from normal and/or luminal epithelial cells, wherein said normal and/or luminal epithelial cells are grown in cholera toxin-free medium comprising FAD2, TGFβRi, and estrogen as described by the present invention.

### DESCRIPTION OF DRAWINGS

FIG. 1: A unique keratin staining signature, Ks20.8^{high}/CD166^{high}/CD117^{low} of ER^{pos} cells is eligible for cell sorting and tracking. (a) Cryostat sections of normal human breast tissue stained with immunoperoxidase against keratin Ks20.8 (left), and K18 (right) and counterstained with hematoxylin. Note the characteristic scattered staining pattern with Ks20.8 against the uniform lineage related staining of luminal cells with K18. Bar=50 μm. (b) Multicolor imaging of normal human breast cryostat sections stained with Ks20.8 (dark grey), ER, CD166, or CD117 (grey) and TO-PRO®-3 Iodide nuclear stain (light grey). Dual dark grey/light grey (left column), grey/light grey (middle column), and merged (right column) exposures clearly reveal coordinate expression of Ks20.8 with ER and CD116, respectively, and dissociation between Ks20.8 and CD117 staining. Bar=25 μm.

FIG. 2: ER^{pos} cells are purified and tracked by sequential CD326/CD271-CD166/CD117 FACS followed by K20.8 staining. (a) Multicolor flow cytometry of uncultured human breast epithelial cells incubated with CD326/CD271/CD166/CD117 and visualized pairwise (left diagrams) to recover luminal cells (CD326^{hi}) and basal cells (CD271^{hi}) and from the luminal gate CD166 positive and CD117 positive cells. Smears of sorted cells were stained (right panel) with either of the markers against basal cells, cytokeratin K14; luminal cells, cytokeratin K18; luminal progenitors, cytokeratin K15; or ER^{pos} cells, cytokeratin Ks20.8 and counterstained with TO-PRO®-3 Iodide nuclear stain. Bar=50 μm. (b) Purity of sorted cells as determined by staining of smears followed by quantification of the percentage of cells stained with either of the markers cytokeratins K14, K18, K15, and Ks20.8 (3×100 cells pr. slide, bars indicate standard deviations). (c) Heatmap representing qRT-PCR analysis of the relative gene expression of lineage markers in sorted basal cells (basal), CD117^{high} luminal cells (CD117), and CD166^{high} luminal cells (CD166) from six different biopsies. Data confirm different transcriptional profiles of the three cell populations. Bars indicate the fold difference of the relative gene expression in log₂ scale.

FIG. 3: Loss of ER^{pos} cells in culture is due to both lack of growth and down modulation of ER expression. (a) T25 flasks in triplicate of basal cells (basal), CD117^{hi}, and CD166^{hi} ER^{pos} luminal cells plated ata clonal density of 10⁴ cells per flask (400 cells/cm²) and stained with hematoxylin after fourteen days in culture. Note that only CD117^{hi} cells are colony forming at clonal density. (b) A higher magnification of cultures started at a higher cell density (1200 cells/cm²) immunoperoxidase stained with Ks20.8 and counterstained with hematoxylin. Positive staining was confined to non-colony forming cells of the CD166^{hi} cultures. Bar=50 μm.

FIG. 4: Relief of TGF-β-mediated negative regulation of growth releases ER^{pos} cells from quiescence. (a) Phase contrast micrographs (upper panel) after 8 days in culture and immunoperoxidase staining with Ks20.8 (middle panel) and ER (lower panel) of primary cultures of CD166^{hi} derived cells in FAD2 (left column) or TGFβR2i (right column). Nuclei are counterstained with hematoxylin. Whereas Ks20.8 positive cells remain quiescent and ER negative on FAD2, they are colony forming and ER^{pos} in TGFβR2i. Bar=50 μm. (b) Quantification of ER^{pos} (closed bar) or Ks20.8 positive (open bar) colony forming units (CFUs) derived from CD166^{hi} cells from three consecutive biopsies (p957, p958, p959) in FAD2 or TGFβR2i. In all three cases TGFβR2i supported colony formation of Ks20.8-/ER-positive cells. (c) qPCR of ER (ESR1), cytokeratin K8 (KRT8), FOXA1, ELF5 and cytokeratin K18 (KRT18) of RNA extracted from cells cultured in FAD2 (open bars), in FAD2 with SB431543 (shaded bars) and in TGFβR2i (solid bars), respectively. Note the collective upregulation of ER and ER associated geneexpression in TGFβR2i. Error bars indicate standard deviation of three technical triplicates. (d) Western blotting of proteins extracted from cells cultured in FAD2 (left lane), in FAD2 with SB431543 (middle lane) and in TGFβR2i (right lane), respectively, incubated with antibodies recognizing phosphorylated SMAD2 (upper panel), SMAD2/3 (second panel), ER (third panel) and β-actin (lower panel). TGFβR2i inhibits pSMAD2 and upregulates ER protein expression.

FIG. 5: TGFβR2i allows efficient expansion of ER^{pos} cells. (a) Population doublings as a function of time in culture calculated by continuous cell number recordings in triplicate cultures before confluency and plating with a fixed number of 4000 cells/cm² per flask at each split. TGFβR2i allows proliferation for six passages, corresponding to fifteen population doublings (open diamond). If RepSox is omitted, the cells cannot be expanded beyond fourth passage (circle). Initial plating on 3T3 feeders extends proliferation to more than ten passages, corresponding to more than 25 population doublings (closed square). hTERT and shp16 transduction in second and third passage, respectively, and subsequent plating at a fixed number of 6000 cells/cm² at each split extended the proliferative capacity even further (cross), and the cells have now been growing for more than 15 passages. (b) Even beyond twenty population doublings, ER^{pos} cells with definitive life span maintain ER and PR expression as shown by immunoperoxidase and hematoxylin staining. Bar=50 μm.

FIG. 6: ER^{pos} cells respond to estrogen. (a) Staining of PR in second passage CD166^{hi} cells deprived of EGF when exposed to vehicle (EtOH) or estrogen (E2). ER^{pos} cells respond to estrogen by increased expression of PR. (b, left) Quantification of cell number in three experiments of triplicate cultures of EGF-deprived, estrogen-stimulated CD166^{hi} cells seeded at 4000 cells/cm² and grown for four days in second (P959) or third passage (P958), or long-term cultured cells (hTERT/shp16) seeded at 6000 cells/cm² in eleventh passage grown for 15 days in the presence of estrogen (dark grey) or vehicle (light grey). In the presence of estrogen a higher cell number is obtained. (b, right) Quantification of total cell number in four experiments of triplicate cultures of CD166^{hi} cells seeded at 5600 cells/cm² on confluent fibroblast feeders and grown for eight days in the presence of estrogen (dark grey) or vehicle (light grey). Second passage cultures derived from two different biopsies, P958 and P959 (first and fourth set of bars) do not respond differently from third passage cultures (second set of bars), and a similar response was observed irrespective of omission of EGF from TGFβR2i prior to co-culture (second set of bars). Omission of EGF throughout the entire experimental period reduced the total cell number, but did not augment the response to estrogen (third set of bars). Error bars indicate standard deviations, and the difference between experimental and control is in all cases significant by two-tailed t-test, p<0.05. (c) Relative expression of PGR and GREB1 normalized to four reference genes GAPDH, HPRT1, TBP, and TFRC assessed by qRT-PCR of 20 ng RNA extracted from second (P957, P959), third passage (P958), or fourteenth passage long-term (hTERT/shp16) cultured ER^{pos} cells exposed to vehicle (EtOH, light grey bars) or estrogen (E2, dark grey bars) for 4 (P958, P959), 13 (hTERT/shp16) or 22 (P957) days, respectively. Transcription of downstream target genes of ER signaling, PGR and GREB1 were in all cases significantly upregulated by estrogen (two-tailed t-test, p<0.05).

FIG. 7: Ks20.8 is a specific marker of a subset of keratin K8 positive cells with strong staining. (a) Serial cryostat sections of a human reduction mammoplasty stained with Ks20.8 (left) and K8 (right) and counterstained in hematoxylin. Whereas the Ks20.8 staining is specific for the scattered pattern within the luminal lineage, K8 exhibits a similar patterned strong staining in addition to moderate staining in the rest of the luminal cells. Bar=50 μm. (b) Multicolor imaging of a cryostat section of a normal human breast acinus stained with K8 (light grey), Ks20.8 (grey) and nuclei (dark grey). Note the overlap in K8^{hi} staining and Ks20.8 staining. Bar=25 μm.

FIG. 8: Ks20.8 positive cells exhibit an elaborated differentiation program. Multicolor imaging of cryostat sections from normal human breast tissue stained with selected mAbs (light grey) from library, Ks20.8 (grey), and nuclei (dark grey). Note the co-staining between the selected mAbs and Ks20.8. Bar=25 μm

FIG. 9: Profiling of basal cells CD117^{high} and CD166^{high} cells. (a) Multicolor imaging of uncultured HBEC sequentially sorted by CD326/CD271 and CD166/CD117 and stained for estrogen receptor (light grey) and nuclei (dark grey). Note that although there is a general unspecific staining of the cytoplasm, staining of nuclei is limited to CD166high cells (bar=25 μm). (b) Bar graph representing qRT-PCR analysis of additional gene expression profiles in sorted basal, CD117high, and CD166high cells from three different biopsies. Each gene expression level was normalized to the mean of four reference gene expressions and then compared to normalized gene expression levels in basal cells. Colored bars indicate the fold difference of the relative gene expression. Error bars represent SEM of three biological samples which were run in triplicate by qPCR.

FIG. 10: Five out of six biopsies show distinct CD166^{high} population. FACS diagrams of uncultured human breast epithelial cells from six biopsies sorted with CD166^{high} and CD117^{low} cells eligible for sorting.

FIG. 11: Representative FACS profiles illustrating the separation of 67LR^{hi}/CD166^{hi} cells from uncultured human breast epithelial cells. (a) A single cell suspension of primary breast epithelial cells stained with CD326, CD166 and 67LR followed by flow cytometry. ER^{pos} human breast epithelial cells sorted with the CD166^{hi}/67LR^{hi} gate. (b) Smears of FACS sorted cells stained with Ks20.8 (left) or keratin K18 (right). Frequency of positive cells (n=3×50 cells per slide±SD).

FIG. 12: PCR analysis of human estrogen receptor signaling. (a) Using a commercially available PCR array containing 84 genes involved in ER activation and its response, qRT-PCR was conducted with RNA samples of ER^{pos} HBEC with or without TGFβR2i. Among them, 20 genes (some of which are labeled in the plot) were highly expressed in the presence of TGFβR2i. Scatter plot graph shows the expression level of each gene in TGFβR2i treated CD166^{high} cells versus DMSO treated CD166^{high} cells. The black line indicates no difference between samples, while the pink lines indicate a two-fold change in gene expression. Two biological replicates were run I qRT-PCT with duplicates of each. (b) qRT-PCR of ESR and PGR expression in hTERT/shp16 transduced ER^{pos} HBEC exposed to vehicle (DMSO), SB431542 only, Repsox only, or TGFβR2i for 7 days. Note that the highest levels of expression were obtained in TGFβR2i. Bars indicate the fold difference of the relative gene expression and error bars represent SD of triplicates in qPCR.

FIG. 13: TG93R2i expression in ER^{pos} HBEC. Flow cytometric analysis of TGFβR2i surface expression in short-term cultured ER^{pos} HBEC, FITC-TGFβR2i (dark grey). FITC conjugated antibody in the absence of TGFβR2i primary antibody (light grey).

FIG. 14: hTERT/shp16 transduced cells remain ERpos and luminal-like. Immunoperoxidase staining of finite lifespan (left column) and hTERT/shp16 trnasduced (right column) ER^{pos} HBEC with ER, keratin K8 and K19 and P63. Cells were counterstained with hematoxylin. (Bar 100 μm).

FIG. 15. Augmented effect of estrogen on ER^{pos} HBEC by long term exposure and omission of EGF. (a) Immunoperoxidase staining of ER^{pos} HBEC grown in TGFβR2i with estrogen in second passage prior to omission of estrogen in the third passage (left column) Exposure to estrogen (right column) downregulated ER and upregulated PR. Cells were counterstained with hematoxylin, (Bar 100 μm). (b) Multicolor imaging of ER^{pos} HBEC in presence of estrogen stained with ER (light grey), PR (grey), and nuclei (dark grey). Cells are either ER^{pos} (asterix), PR^{pos} (arrowhead), ER^{pos}/PR^{pos} (arrows), or ER^{neg}/PR^{neg} (not labeled), (Bar 100 μm).

FIG. 16. Stable luminal phenotype. CD166^{high}/CD117^{low} hTERT/shp16 cells cultured in FAD2+SB431542+RepSox (TGFβR2i) were sorted by FACS as CD146^{high} in passage 6 and 11. After culture until passage 22 the cells were sorted by FACS as EpCAM^{high}/CD146^{high}/CD117^{high} (P7 gate) and switched to FAD3+SB431542+RepSox. Subsequent cultures were split at up to 6000 cells/cm2, and their luminal phenotype is stable beyond passage 35.

FIG. 17. Stable luminal phenotype. (A) B506 (hTERT/shp16, CD166hi/CD117lo) passage 6 cells are sorted with CD146 to yield F1221-P4. (B) F1221-P4 (hTERT/shp16, CD166hi/CD117lo/CD146hi) passage 11 cells were sorted with CD146 and termed F1223-P4. (C) F1223-P4 (CD166hi/CD117lo/CD1462xhi) passage 14 cells were sorted with CD146 and termed F1224-P4.

### DETAILED DESCRIPTION OF THE INVENTION

Definitions

The suffix ‘high’ and ‘low’ or ‘hi’ and ‘lo’, as used herein describes the relative level of a marker (e.g. CD117^{high} and CD117^{low}). It is understood that for a given marker, the attributed suffix ‘high’ or ‘low’ has clear meaning to the skilled person. For instance, ‘low’ or ‘lo’ (e.g. CD117^{low}) means that the marker is undetectable, absent or low, at least lower than an intermediate expressing population. Likewise, ‘high’ or ‘hi’ (e.g. CD117high) means that expression is detectable, and/or have higher intensity of staining than ‘low’ cells and possibly higher than intermediate staining populations. In the case of KS20.8, Ks20.8^{high} cells refer to cells to which the Ks20.8 antibody can bind at a detectable level, while Ks20.8^{low} cells refer to cells to which the Ks20.8 antibody cannot bind at a detectable level.

The term “ER^{pos}” or “estrogen receptor positive” or “ER positive” as used herein, refers to a cell in which the estrogen receptor protein is present in levels which are detectable using immunocytochemistry or Western blot. In addition the cell should exhibit an estrogen receptor growth response e.g. downstream activation of transcripts such as the progesterone receptor.

The term “cell strain” or “strain” as used herein as used herein describes a population of cells which derive from a primary cell from a multicellular organism which has not been immortalized and which can be grown for a limited period in vitro. Cell strains of the present invention comprises CD166^{high} cells which have been cultured on FAD2 medium supplemented with TGFβR2i in Primaria flasks. Typically cell strains of the present invention remain ER positive for 1-6 passages. Transfection of cell strains of the present can yield cell lines as described by the present invention.

The term “cell strain with extended lifespan” or “strain with extended lifespan” as used herein describes a population of cells which derive from a primary cell from a multicellular organism which has not been immortalized and which can be grown for a limited period in culture. Cell strains with extended lifespan may be obtained by transient co-culturing with feeder cells such as irradiated NIH-3T3 feeders in presence of BBMYAB medium and subsequently sorted by FACS to isolate CD326^{high}/CD271^{high} cells prior to plating and passaging in TGFβR2i culture. A cell strain with extended lifespan as described by the present invention may derive from a primary cell having specific properties or characteristics. Cell strains with extended lifespan are cells that have been adapted to culture but, unlike cell lines, have a finite division potential. A cell strain with extended lifespan of the present invention may stop dividing and/or loose the ER^{pos} phenotype after 20-30 population doublings when cultured at appropriate conditions.

The term “immortalized” as used herein may refer to a cell, a cell strain, a cell line or a cell population which would normally not proliferate indefinitely but have evaded normal cellular senescence and instead can keep undergoing division. Cells can be immortalized due to e.g. mutations, which can occur spontaneously or which can be introduced by genetic means, e.g. as described herein.

The term “BBMYAB medium” as used herein refers to a medium comprising BBM without HEPES (DM EM/F-12 (Dulbecco's Modified Eagle Medium/Nutrient Mixture F-12, Life Technologies), 1 μg/ml hydrocortisone (Sigma-Aldrich), 9 μg/ml insulin (Sigma-Aldrich), 5 μg/ml Transferrin (Sigma-Aldrich), 5.2 ng/ml Na-Selenite (BD Industries), 100 μM ethanolamine (Sigma-Aldrich), 20 ng/ml basic fibroblast growth factor (bFGF) (PeproTech), 5 nM amphiregulin (R&D Systems), (Pasic, L., et al. Sustained activation of the HER1-ERK1/2-RSK signaling pathway controls myoepithelial cell fate in human mammary tissue. Genes & Dev 25, 1641-1653 (2011)) with the addition of 1.8×10⁻⁴ M adenine (Sigma-Aldrich), 10 μM Y-27632 (Sigma-Aldrich) and 20 μl/ml B27 (Life Technologies). Other similar media compositions as described in the section “Culture medium B” may be used by the present invention as replacement of the BBMYAB media.

The term “Y-27632” as used herein refers to a highly potent, cell-permeable, selective ROCK (Rho-associated coiled coil forming protein serine/threonine kinase) inhibitor. Kᵢ=140 nM for p160^{ROCK}. Y-27632 also inhibits ROCK-II with equal potency. The inhibition is competitive with respect to ATP.

The term “cell line” or “cell strain” as used herein, refers to a population of cells derived from a single cell which would normally not proliferate indefinitely but, due to genetic modification have evaded normal cellular senescence and instead can keep undergoing division if cultured at appropriate conditions. The genetic modifications required for immortality can occur naturally or be intentionally induced for experimental purposes e.g. insertion of a telomerase reverse transcriptase gene as well as shRNA targeting cyclin dependent kinase inhibitor p16. Cell lines are cells that have been adapted to culture indefinite at appropriate conditions.

The term “primary cell” as used herein refers to a cell which derives directly from the parent tissue. Primary cells have the same karyotype and chromosome number as those in the original tissue.

The term “progenitor cell” as used herein, refers to a biological cell that, like a stem cell, has a tendency to differentiate into a specific type of cell, but is already more differentiated than a stem cell and has started the differentiation towards its “target” cell. Upon appropriate stimuli normal cells can alternate between the progenitor cell state and the stem cell state.

The term “population doubling” or “population doubling level” or “PDL” as used herein, refers to the total number of times the cells in the population have doubled since their primary isolation in vitro. This is usually an estimate rounded off to the nearest whole number. Population doubling is calculated using the following formula:

$$PD = \frac{Log ( \frac{N}{{X}_{0}} )}{Log ( 2 )} + X$$

N=final cell yield (number of cells at the end of the growth period, also known as UCY)

X₀=cell inoculum (number of cells plated in the flask, also known as I)

PD=number of total population doublings

X=the doubling level of the inoculum used to initiate the subculture being quantitated. e.g. 100,000 (=X₀) cells are seeded, grown for a number of days and counted: 800,000 (=N). These cells have been grown through several passages and have thus far made 12 population doublings (=X). From last calculation and until now, the cells have made

$$\frac{Log ( \frac{800000}{100000} )}{Log ( 2 )} + 12 PD = 15 PD$$

This method is the same as the “3.32 (Log(UCY)−Log(I))+X” method, only rewritten, as

$$\begin{matrix}3.32 = \frac{1}{Log ( 2 )} and & 1 ) \\ Log ( \frac{N}{{X}_{0}} ) = Log ( N ) - Log ( {X}_{0} ) & 2 )\end{matrix}$$

We use the equation initially mentioned, as 3.32 is an approximation and not the exact value of

$$\frac{1}{Log ( 2 )} .$$

A more complete discussion of population doubling calculations is provided by L. Hayflick (1973) Tissue Culture Methods and Applications, P. F. Kruse, Jr. and M. K. Patterson, Jr. eds., p. 220 (Academic Press, New York).

The term “tumorigenic cell” as used herein refers to a cell capable of causing a tumor in a tissue or culture.

The term “agent” or “candidate agent” as used herein, refers to any molecule. In an embodiment an “agent” or “candidate agent” is able to reduce proliferation and/or survival of a tumorigenic cell as described in the present invention. Examples of agents may be, but are not limited to peptides, proteins, siRNAs, DNA, cofactors, small molecules e.g. specialized/secondary or primary metabolites from any biological origin. An agent of the present invention may also be a synthetic or biosimilar peptide, protein, siRNA, DNA, cofactor, small molecule e.g. specialized/secondary or primary metabolite made through synthetic or semi-synthetic synthesis and/or by heterologous methods.

The term “metastasis”, or “metastatic disease”, as used herein, refers to the spread of a cancer or disease from one organ or part to another, not directly connected with it.

The term “providing a cell”, as used herein, refers to providing a cell such as a breast cell. A non-limiting list of how a cell may be provided comprise providing a cell from a mammoplasty, such as a mammoplasty for cosmetic reasons, providing a cell from any breast tissue, such as from non-tumorigenic tissue and such as from tumorigenic tissue. Epithelial cells may be purified by collagenase treatment followed by freezing. Collagenase treatment may yield lumps of epithelia with associated collagen tissue (referred to as organoids) which may be frozen for later use. In most of the experiments described in the present invention thawed organoids have been used. Sorted cells (e.g. by FACS) may as well be frozen and later used for culturing. In our experience epithelium, i.e. organoids or sorted cells may keep several years in the freezer.

The term “anti-cytokeratin 20 antibody” or “Ks20.8” as used herein refers to an anti-cytokeratin 20 antibody. The clone Ks20.8 was originally raised against keratin (Moll et al., 1992). The inventors have further shown that Ks20.8 is a novel marker, which can be used to identify ER-positive cells, but also cells that no longer express the ER but have the ability or have had the ability to express the ER.

ER^{pos} Cell Line

In the present invention represents a solution for long-term culturing of an ER^{pos} cell line which originates from a primary cell isolated from breast tissue. Long-term culturing of an ER^{pos} cell line constitutes an important molecular tool to study the evolution of breast cancer and may constitute an important assay for identifying agents for prevention or treatment of breast cancer. In spite of several decades of intense research of breast cancer, ER^{pos} normal breast epithelial cell lines have not yet been available. Thus the present invention represents the first ER^{pos} normal breast epithelial cell line which provides a, in principle, near infinite supply of ER expressing breast epithelial cells for in culture assays without being dependent on a supply of mammary epithelial cells from biopsies. The inventors have surprisingly shown that ER positive cell lines can be cultured for more than 15 passages and have established multiple clones.

Thus a major aspect of the present invention relates to an immortalized estrogen receptor positive cell line, wherein said cell line has a CD326^{high}/CD271^{low} phenotype. In a preferred embodiment, the cell line of the present invention is capable of responding to estrogen.

The inventors have developed a non-invasive method for characterizing the cell line of the present invention based on the presence and levels of specific surface proteins. The cell line of the present invention has a distinct surface marker phenotype. In an embodiment the cell line of the present invention has a CD326^{high}/CD271^{low} phenotype. In another embodiment the cell line of the present invention has a CD166^{high}/CD117^{low} and/or CD166^{low}/CD117^{high} phenotype. In a further embodiment the cell line of the present invention has an anti-Ks20.8^{high} phenotype. In a preferred embodiment the cell line of the present invention has a CD326^{high}/CD271^{low}/CD166^{high}/CD117^{low} phenotype. In a most preferred embodiment the cell line of the present invention has a CD326^{high}/CD271^{low}/CD166^{high}/CD117^{low} /anti-Ks20.8^{high}. Primary cells with the above mentioned phenotypes may be used to generate the cell line of the present invention. Cells with other phenotypes may be used as well under appropriate conditions. In an embodiment the cell line of the present invention originates from a progenitor cell with a CD326^{high}/CD271^{low}/CD166^{low}/CD117^{high} and/or a CD326^{high}/CD271^{low}/CD166^{high}/CD117^{low} wphenotype.

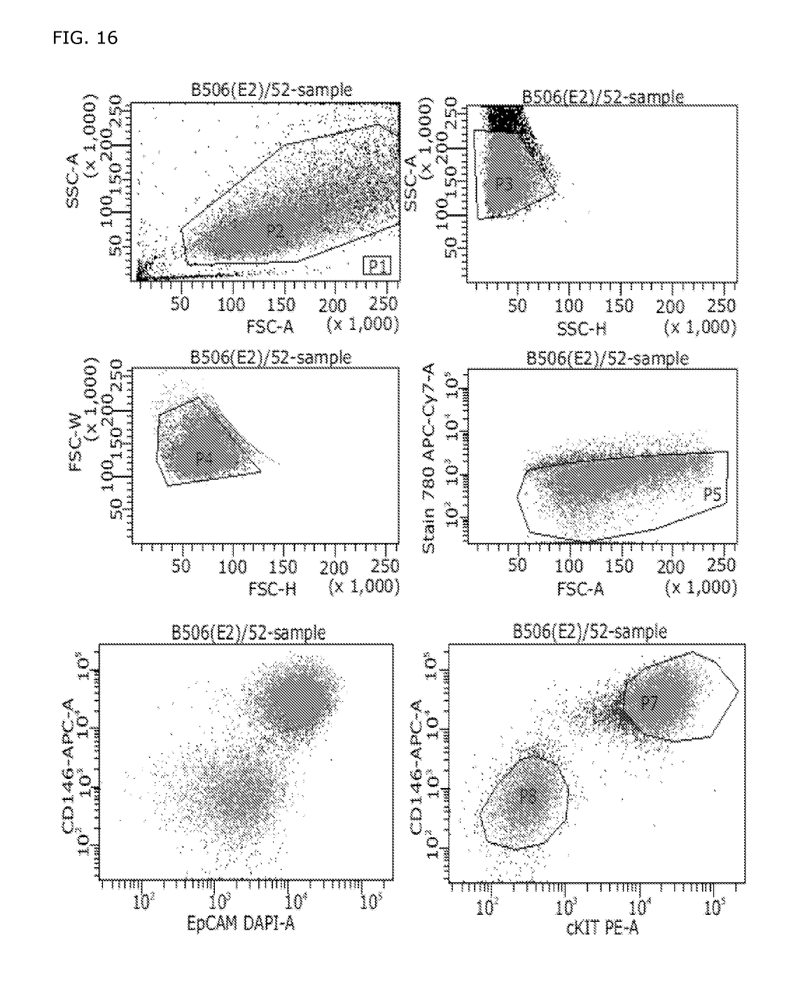

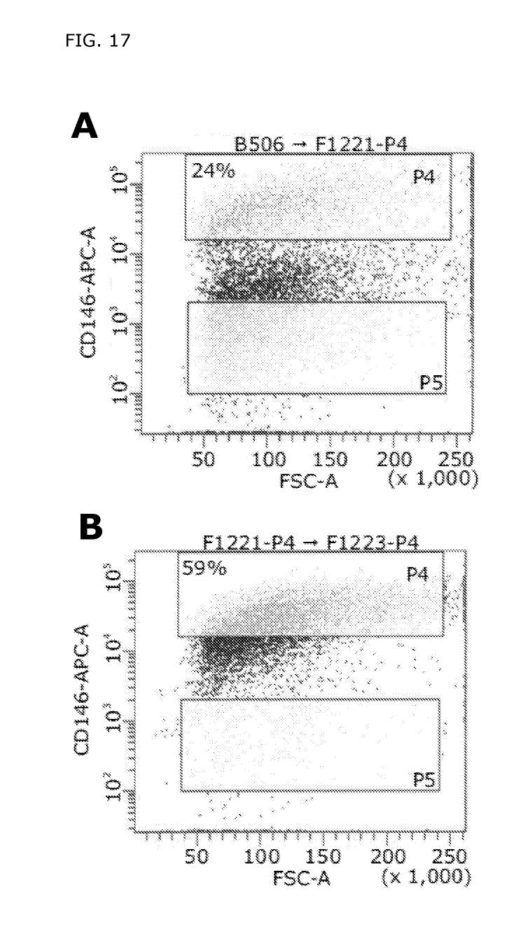

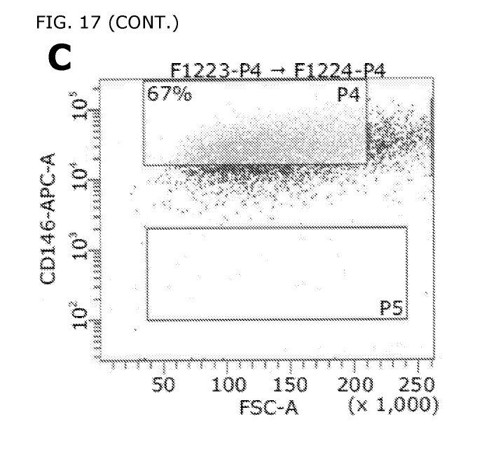

In order to provide a stable phenotype, i. e. a cell line that remains luminal and does not drift towards a basal (p63+, K14+) phenotype, additional sortings by FACS may be performed. For example, sorting can be performed in passage 6 of hTERT/shp16 CD166^{high}/CD177^{low} as CD146^{high} (P1H12 1:500), again in passage 11 and then in passage 14 with the exclusion of larger cells (forward scatter) found by immunostaining to comprise K14+ cells. These were stable at least to passage 22 (see example “stable luminal phenotype” and FIGS. 16 and 17). An alternative approach is a third sorting for instance in passage 22 as EpCAM^{high} (EBA-1, 1:20), CD146^{high} (P1H12, 1:20) and CD117^{high} (104D2, 1:20). The sorted cells have been grown beyond passage 35 and the phenotype is still stable. The sorted cells propagated in FAD2 or in FAD2 without EGF and with estrogen (10⁻⁸ M) maintain a luminal phenotype, defined as ER+, Ks20.8+, K8+, K19+, p63- and essentially K14-, and are still responsive to estrogen, for instance by induction of PR (progesterone receptor).

Cells or cell lines according to the invention can be identified using a number of markers. In one embodiment, the marker is Ks20.8. In another embodiment, the cell line has a CD326^{high}/CD271^{low}/CD166^{high}/CD117^{low} and/or CD326^{high}/CD271^{low}/CD166^{high}/CD117^{low} phenotype and the marker is selected from PR (progesterone receptor), APβ, GATA3, Bcl2, CDw75, N-cadherin and the laminin receptor 67LR.

A cell may be immortalized by introduction of one or more genes which enables the cell to evade senescence and instead the cell can continue to proliferate under appropriate conditions. Thus in an embodiment of the present invention the cell line has been immortalized by genetic modification. The present inventors demonstrate herein that insertion of a telomerase reverse transcriptase can immortalize the ER^{pos} cells of the present invention. Thus in an embodiment the cell line of the present invention has been immortalized by insertion of a telomerase reverse transcriptase. In another embodiment the cell line of the present invention has been immortalized by insertion of a human telomerase reverse transcriptase (hTERT) gene and/or a similar gene, such as a gene which encodes a telomerase reverse transcriptase which is at least 75% identical to the gene product of hTERT, such as at least 85% identical to the gene product of hTERT, such as at least 90% identical to the gene product of hTERT, such as at least 95% identical to the gene product of hTERT, such as at least 96% identical to the gene product of hTERT, such as at least 97%, identical to the gene product of hTERT, such as at least 98% identical to the gene product of hTERT, such as at least 99% identical to the gene product of hTERT, such as at least 100% identical to the gene product of hTERT. In an embodiment the gene encoding hTERT comprise at least one silent mutation. Silent mutations are base substitutions that result in no change of the amino acid or amino acid functionality when the altered messenger RNA (mRNA) is translated. For example, if the codon AAA is altered to become AAG, the same amino acid—lysine—will be incorporated into the peptide chain.

In an embodiment the cell line of the present invention has been immortalized by insertion/transfection of a shRNA P16 gene and/or a similar gene, such as a gene which encodes a shRNA P16 which is at least 75% identical to the gene product of shRNA P16, such as at least 85% identical to the gene product of shRNA P16, such as at least 90% identical to the gene product of shRNA P16, such as at least 95% identical to the gene product of shRNA P16, such as at least 96% identical to the gene product of shRNA P16, such as at least 97%, identical to the gene product of shRNA P16, such as at least 98% identical to the gene product of shRNA P16, such as at least 99% identical to the gene product of shRNA P16, such as at least 100% identical to the gene product of shRNA P16. In an embodiment the gene encoding shRNA P16 comprise at least one silent mutation. Silent mutations are base substitutions that result in no change of the amino acid or amino acid functionality when the altered messenger RNA (mRNA) is translated. For example, if the codon AAA is altered to become AAG, the same amino acid—lysine—will be incorporated into the peptide chain.

In a most preferred embodiment the cell line of the present invention has been immortalized by insertion of a human telomerase reverse transcriptase (hTERT) gene and/or shRNA targeting P16 gene.

Other genetic modifications which can lead to immortalization of the cells of the present invention may be used as well. In an embodiment any known method for immortalizing a cell can be used to immortalize the cell line of the present invention.

The present inventors have shown that primary breast epithelial progenitor or stem cells are capable of generating a cell line of the present invention. Thus in an embodiment of the present invention the cell line is derived from a primary breast epithelial progenitor or stem cell. The inventors have further shown that primary breast epithelial luminal progenitor cells are very suitable for generating a cell line of the present invention. Thus in a preferred embodiment the cell line of the present invention is derived from a primary breast epithelial luminal progenitor cell. The inventors have identified several cell types which may be used as a starting point for yielding an immortalized estrogen receptor positive cell line, wherein said cell line has a CD326^{high}/CD270^{low} phenotype. Thus in a more preferred embodiment the cell line of the present invention is derived form a primary breast epithelial luminal progenitor cell with a CD326^{high}/CD271^{low}/CD166^{high}/CD117^{low}/Ks20.8^{high} phenotype.

The present invention further relates to methods for isolating cells which may be used as starting point to generate the cell line of the present invention. Thus in an embodiment the cell line of the present invention is derived from a primary cell isolated according to isolation methods provided by the present invention. See for example the section “Identification and isolation of primary breast cells” of the present invention.

The present invention further relates to a culturing medium A for said cell line of the present invention. Thus in an embodiment the cell line of the present invention is cultured in a culture medium A according to the present invention. See for example the section “Culture medium A” of the present invention.

The cell line of the present invention is capable of being maintained through a near in principle indefinite number of population doublings under appropriate conditions. The inventors have shown that the cell line of the present invention can be maintained through more than 25 population doublings, while maintaining ER^{pos}. Thus in an embodiment the cell line of the present invention remains estrogen receptor positive for at least 25 population doublings, such as at least 30 population doublings, such as at least 40 population doublings, such as at least 50 population doublings.

ER^{pos} Cell Strains With Extended Lifespan

A major difference between an ER^{pos} cell line culture and an ER^{pos} cell strain with extended lifespan culture is the life span of the culture. As shown by the inventors the lifespan is extended by co-culturing cell strains together with feeder cells, such as NIH-3T3 fibroblasts. While both cell lines and cell strains with extended lifespan remain ER positive for more than a few population doublings, cell lines are immortalized and have an indefinite lifespan while cell strains with extended lifespan have a definite lifespan. To immortalize a cell to generate a cell line a telomerase reverse transcriptase gene and shp16 can be inserted as shown by the present invention. However, such genetic modification may create a cell line which behaves very similarly, in terms of e.g. expression profile, compared to the primary cell from which the cell line is derived. It may therefore be desirable to avoid immortalization by genetic modification but instead be able to create a culture which can mimic a more natural “cellular behavior” compared to an immortalized cell line,An alternative hereto may be to create non-immortalized cell cultures. Non-immortalized cell cultures with a definite lifespan are referred to as cell strains with extended lifespan when co-cultured with feeder cells as described in the present invention. The present invention discloses for the first time the creation of long term cultures of ER^{pos} cell strains with extended lifespan from primary breast epithelial cells.

Thus an aspect of the present invention relates to a method of generating an estrogen receptor positive cell strain with extended lifespan from an isolated breast epithelial cell, the method comprising the steps of:
a. isolating a breast epithelial cell as described herein; and
b. culturing said isolated cell in presence of at least one feeder cell in culture medium B as described herein,
c. isolating a cell with a CD326^{high}/CD271^{high} and/or CD326^{high}/CD271^{low} phenotype, and
d. culturing said isolated cell of step c in culture medium A as described herein,

wherein said isolated cell generates an estrogen receptor positive cell strain with extended lifespan capable of responding to estrogen.

In some embodiments, cells with a CD326^{high}/CD271^{high} and/or CD326^{high}/CD271^{low} phenotype can be identified using a number of markers, such as Ks20.8, PR (progesterone receptor), AP2β, GATA3, Bcl2, CDw75, N-cadherin and the laminin receptor 67LR.

It will be understood that cells with a CD326^{high}/CD271^{high} and/or CD326^{high}/CD271^{low} phenotype can be isolated by negative selection, i.e. by selecting the cells that have a CD326^{low}/CD271^{high} and/or CD326^{low}/CD271^{low} phenotype.

Upon substitution of EGF with amphiregulin a higher level of ER and PR is observed. Hence in an embodiment EGF is substituted with an EGF-like compound. In an embodiment EGF is substituted with amphiregulin. In some embodiments, amphiregulin is added at a concentration of between 1 and 10 nM, such as between 2 and 8 nM, such as between 3 and 7 nM, such as between 4 and 6 nM, such as about 5 nM. Small modifications and substitutions may further optimize the media described in the present invention.

A further improvement of the culture medium A, described in detail in the section entitled “Culture medium A/TGFβR2i medium”, is the substitution of epidermal growth factor with amphiregulin as described above and the exclusion of hydrocortisone and cholera toxin. This medium (FAD3) is moreover applicable to tumor cell lines, such as MCF7, thus allowing an unprecedented direct comparison between normal-derived and tumor-derived estrogen receptor-positive cells. Likewise, primary CD166^{high}/CD117^{low} cells maintain ER expression and grow better in FAD3 than in FAD2. Without being bound by theory, the lifespan of normal cells may also be improved in FAD3.

The inventors have shown that the cell strain with extended lifespan of the present invention can remain ER^{pos} for at least 15 population doublings. Surprisingly, the inventors have demonstrated how initial co-culturing with NIH-3T3 feeder cells is able to increase the number of population doublings through which the cell strain with extended lifespan of the present invention remains ER^{pos}. For example the inventors have shown that the cell strain with extended lifespan of the present invention remained ER^{pos} through at least 10 population doublings. In an embodiment the cell strain with extended lifespan of the present invention remains ER^{pos} through at least 10 population doublings, such as at least 15 population doublings, such as at least 20 population doublings, such as at least 25 population doublings.

The present inventors have demonstrated long-term culturing of a cell strain with extended lifespan by culturing on feeder cells comprising culture medium B, as described herein, in the primary culture followed by culturing in Primaria flasks using FAD2 containing medium as described herein. Thus in an embodiment the cell strain with extended lifespan of the present invention is initially cultured in the presence of a feeder cell.

In an embodiment the feeder cell of the present invention comprises a fibroblast cell. The cell strain with extended lifespan is in an embodiment co-cultured with murine NIH-3T3 fibroblasts feeder cells. In an embodiment the feeder cells of the present invention comprise murine fibroblast cells, such as irradiated NIH-3T3 cells, wherein the cells of the present invention grow in flat islets. In an embodiment the feeder cells of the present invention comprise human fibroblast cells, wherein the cells of the present invention recapitulates polarized 3-D epithelial organization reminiscent of the in situ phenotype. In an embodiment the cell strain with extended lifespan described by the present invention remains ER^{pos} through at least 10 population doublings, such as at least 15 population doublings, such as at least 20 population doublings, such as at least 25 population doublings 25 population doublings by co-culturing feeder cells, such as murine fibroblast such as NIH-3T3 murine fibroblast cells.

Another aspect of the present invention relates to a method of growing and/or proliferating estrogen receptor positive human breast epithelial cells, wherein said cells remain estrogen receptor positive after more than four population doublings, said method comprising the steps of:
a. providing a luminal progenitor cell isolated from human breast epithelial according to the present invention; and
b. growing said cell in in culture medium A or in a culture medium comprising;
   i. hydrocortisone;
   ii. cholera toxin;
   iii. adenine;
   iv. Y-27632;
   v. fetal bovine serum;
   vi. epidermal growth factor or amphiregulin;
   vii. insulin; and
   viii. an inhibitor of a TGF-β type I receptor as described in the present invention.

The present invention provides several different culture conditions for cell strains with extended lifespan. The inventors have also shown that the culture conditions strongly influence the lifespan of the cell strain with extended lifespan despite all cell strains with extended lifespan being derived from the same type of primary cell.

ER^{pos} Cell Strains

A major difference between an ER^{pos} cell strains with extended lifespan and an ER^{pos} cell strains is the life span and the duration of the ER expression in the culture. The inventors have shown that cell strains typically remain ER positive for 3 to 6 passages when grown on FAD2 supplemented with TGFβR2i in Primaria flasks. The inventors have further observed a variation between biopsies. Thus an aspect of the present invention relates to a method of generating an estrogen receptor positive cell strain from an isolated primary breast epithelial cell, the method comprising the steps of:
a. isolating a primary breast epithelial as described by the present invention; and
b. culturing said isolated cell in culture medium A as described by the present invention;

wherein said isolated cell generates an estrogen receptor positive cell strain capable of responding to estrogen.

3-D Epithelial Organization Phenotype

Surprisingly, the present inventors have further shown that co-culturing the cell line, cell strain, and/or cell strain with extended lifespan of the present invention with human fibroblast feeder cells in culture medium B, as described herein, recapitulates polarized 3-D epithelial organization reminiscent of the in situ phenotype, including ER expression. Specifically the present inventors have shown that CD105^{high} human breast fibroblast feeder cells results in three-dimensional development of the cell line, cell strain, and/or cell strain with extended lifespan of the present invention. The inventors have further shown that CD105^{high}/CD26^{low} and CD105^{low}/CD26^{high} fibroblasts, which represent end- and duct fibroblasts, respectively, yields different morphogenenic response,

Thus in an embodiment the feeder cells of the present invention comprises human fibroblast cells, wherein the cell line, cell strain, and/or cell strain with extended lifespan and/or clones thereof as described by the present invention recapitulates 3-D epithelial organization reminiscent of the in situ phenotype when cultured in culture medium B as described herein. In an embodiment the feeder cells of the present invention comprises CD105^{high}/CD26^{low} and/or CD105^{low}/CD26^{high} human fibroblasts. In an embodiment the feeder cells of the present invention comprise CD105^{high} human breast fibroblast. In an embodiment the cell line, cell strain, and/or cell strain with extended lifespan and/or clones thereof described by the present recapitulates 3-D epithelial organization reminiscent of the in situ phenotype when cultured in culture medium B with or without estrogen. In an embodiment the cells which are organized in a 3-D phenotype are ER^{pos}.

Non-Invasive Methods for Identification and Isolation of Primary Breast Cells

The current method for identifying an ER^{pos} cell relies on detection of the ER, which is found in the nucleus of cells. Invasive methods such as immunostaining/cytochemistry are often required to detect intracellular receptors. Alternatively, identification of ER^{pos} cells can be confirmed by observing a growth response or downstream activation of transcripts such as the progesterone receptor. None of these methods are non-invasive or compatible with high throughput cell sorting systems such as FACS, which primarily rely on detection of surface associated proteins. Primary cell types may be identified and classified on the basis of cell surface proteins e.g. Classification Determinants. Today there are no protocols for identifying ER^{pos} cells based on cell surface proteins. Consequently, much research relies on ER^{pos} cells, which is conducted on freshly isolated small pieces of breast tissue. Considering the importance of ER^{pos} cells in breast cancer research it is therefore a desirable development of a reliable protocol for isolation of these cell types.

The inventors of the present invention have found markers for identifying primary cells from breast tissue which can be used to establish a long-term culture of ER^{pos} cells as described in the present invention. As shown in the examples the inventors have used FACS for easy sorting of individual cells, however, any fluorescent based automatic and/or manual sorting techniques could be used instead.

Thus an aspect of the present method relates to a method of isolating a primary breast epithelial cell which is capable of establishing the estrogen receptor positive cell line as described in the present invention and/or the estrogen receptor positive cell strain with extended lifespan described in the present invention and/or the cell strain as described by the present invention, said method comprising the steps of:
a. providing a sample of breast epithelium cells; and
b. isolating a primary cell with a CD326^{high}/CD271^{low} phenotype,

thereby isolating a primary breast epithelial cell capable of yielding a cell line as described in the present invention (e.g. in the “ER^{pos} cell line” section) and/or cell strain with extended lifespan as described in the present invention (e.g. in the “ER^{pos} cell strains with extended lifespan” section) and/or the cell strain as described by the present invention (e.g. in the “ER^{pos} cell strains” section.

The inventors found that the growth conditions of the present invention yield ER^{pos} cells from ER^{pos} cells (CD166^{high}/CD117^{low} phenotype), but surprisingly also from luminal ER^{neg} progenitors (CD166^{low}/CD117^{high} phenotype). Thus in an embodiment said isolated primary cell described by the present invention further has a CD166^{high}/CD117^{low} or CD166^{low}/CD117^{high} or CD326^{high}/CD271^{low} phenotype. In a preferred embodiment said isolated cell described in the present invention has a CD166^{high}/CD117^{low} phenotype.

The inventors have further shown that Ks20.8 is a novel ER^{pos} cell marker. Thus in an embodiment said isolated primary cell further has a Ks20.8^{high} phenotype.

Another aspect of the present method relates to a method of isolating a primary breast epithelial cell which is capable of establishing the estrogen receptor positive cell line as described in the present invention and/or the estrogen receptor positive cell strain with extended lifespan as described by the present invention, said method comprising the steps of:
a. providing a sample of breast epithelium cells; and
b. isolating a primary cell with a Ks20.8^{high} phenotype,

thereby isolating a primary breast epithelial cell capable of yielding a cell line as described in the present invention (e.g. in the “ER^{pos} cell line” section) and/or cell strain with extended lifespan as described in the present invention (e.g. in the “ER^{pos} cell strain” section).

In an embodiment the isolated cell further has a CD326^{high}/CD271^{low} phenotype. In an embodiment said isolated primary cell described by the present invention further has a CD166^{high}/CD117^{low} or CD166^{low}/CD117^{high} phenotype. In a preferred embodiment said isolated cell described in the present invention has a CD166^{high}/CD117^{low} phenotype.

Another aspect of the present method relates to a method of isolating a primary breast epithelial cell which is capable of establishing the estrogen receptor positive cell line as described in the present invention and/or the estrogen receptor positive cell strain with extended lifespan as described by the present invention, said method comprising the steps of:
a. providing a sample of breast epithelium cells; and
b. isolating a primary cell with a CD166^{high}/CD117^{low} or CD166^{low}/CD117^{high} phenotype,

thereby isolating a primary breast epithelial cell capable of yielding a cell line as described in the present invention (e.g. in the “ER^{pos} cell line” section) and/or cell strain with extended lifespan as described in the present invention (e.g. in the “ER^{pos} cell strain” section).

In an embodiment the isolated primary cell further has a CD326^{high}/CD271^{low} phenotype. Thus in an embodiment said isolated primary cell further has a Ks20.8^{high} phenotype.

In an embodiment the breast epithelial cells are breast duct epithelial cells and/or breast lobules epithelial cells. In an embodiment the sample of breast epithelial cells of the present invention derive from a mammoplasty, such as a mammoplasty for cosmetic reasons.

Another aspect of the present invention relates to a method of isolating a primary breast epithelial cell which can be used to establish a long-term culture of ER^{pos} cell as described in the present invention, said method comprising the steps of:
a. providing breast epithelium;
b. collecting a primary cell with a CD326^{high}/CD271^{low} phenotype,

thereby isolating a primary breast epithelial cell capable of yielding a cell line according as described in the present invention. In an embodiment said collected cell further has a CD166^{high}/CD117^{low} or CD166^{low}/CD117^{high} phenotype. In another embodiment collected cell said cell further has a Ks20.8^{high} phenotype or a Ks20.8^{low} phenotype. In an embodiment Ks20.8^{high} and/or Ks20.8^{low} phenotype is determined by the anti-cytokeratin 20 antibody clone Ks20.8. Several anti-cytokeratin 20 antibody clones Ks20.8 are commercially available. Identifying the best suited clone(s) for performing the invention may require routine optimization, which the skilled person knows how to perform.

In some embodiments, cells with a CD326^{high}/CD271^{high} and/or CD326^{high}/CD271^{low} phenotype according to the invention can be identified using a number of markers, such as Ks20.8, PR (progesterone receptor), APβ, GATA3, Bcl2, CDw75, N-cadherin and the laminin receptor 67LR.

It will be understood that cells with a CD326^{high}/CD271^{high} and/or CD326^{high}/CD271^{low} phenotype can be isolated by negative selection, i.e. by selecting the cells that have a CD326^{low}/CD271^{high} and/or CD326^{low}/CD271^{low} phenotype.

In an embodiment the collected cell as described herein is from a mammal, such as a mammal selected from the group comprising of human beings, mice, rats, and/or rabbits. In a preferred embodiment the collected cell is from a human being.

In another embodiment the collected cells of the present invention are collected using FACS sorting.

Culture Medium A/TG93R2i Medium

An aspect of the present invention relates to a culture medium A, which is capable of releasing ER^{pos} breast epithelial cells from their growth restraint while maintaining the ER^{pos} phenotype in vitro through several population doublings. Earlier attempts on culturing ER^{pos} cells in vitro have resulted in loss of ER expression after very few population doublings. Surprisingly, growth conditions of the present invention also yielded ER^{pos} cells from luminal ER^{neg} progenitors. This indicates that at least two different cell types ER^{pos} and ER^{neg} cells can be used as starting point for creating a long term ER^{pos} in vitro culture. Culture medium A may be referred to as TGFβR2i medium in the present invention.

TGF-β Type I Receptor Inhibitors

The present inventors have found that addition of one or more TGF-β type I receptor inhibitors are important components of the culture medium A of the present invention. Thus in an aspect of the present invention relates to a cell culture medium A for inducing and/or maintaining an estrogen receptor positive phenotype in a primary breast epithelial cell or a cell line as described by the present invention, wherein the culture medium A comprises; an inhibitor of a TGF-β type I receptor. In an embodiment said inhibitor of a TGF-β type I receptor comprises an inhibitor of a TGF-β type I receptor activin receptor-like kinase and/or an inhibitor of a TGF-β type I receptor activin receptor-like kinase autophosphorylation. In another embodiment said inhibitor of a TGF-β type I receptor comprises one or more compounds selected from the group comprising of SB431542, RepSox, and/or SD208. In an embodiment said inhibitor of a TGF-β type I receptor comprises RepSox. In another embodiment any inhibitor of TGF-β type I receptor may be used, such as an inhibitor of TGFβ type I receptor kinase (ALK) 5 (ALKS), ALK4, and/or ALK7 and or such as an inhibitor of autophosphorylation of ALKS. In another embodiment said inhibitor of a TGF-β type I receptor comprises one or more compounds selected from the group comprising of TGF-βR2i (a combination of SB431542 and RepSox), SD208, LDN-212854, LY 2157299, A 83-01, SD 208, D 4476, LY 364947, GW 788388, SB 505124, SB 525344, LY 2109761. In another embodiment said inhibitor of a TGF-β type I receptor comprises SB431542. In another embodiment said inhibitor of a TGF-β type I receptor comprises a combination of SB431542 and RepSox. In an embodiment said inhibitor of a TGF-β type I receptor comprises SB431542. In an embodiment said inhibitor of a TGF-β type I receptor comprises RepSox. In an embodiment said inhibitor of a TGF-β type I receptor comprises SD208. In another embodiment said inhibitor of a TGF-β type I receptor comprises a combination of SB431542 and RepSox, wherein the concentration of SB431542 is from 0.1 μM-1000 μM SB431542, such as from 1 μM-100 μM SB431542, such as from 1 μM-50 μM SB431542, preferably such as from 1 μM-30 μM SB431542, more preferably such as from 3 μM-20 μM SB431542, most preferably such as around 10 μM SB431542 and wherein the concentration of RepSox is from 0.5 μM-500 μM RepSox, such as from 5 μM-100 μM RepSox, such as from 5 μM-50 μM RepSox, preferably such as from 25 μM-150 μM RepSox, more preferably such as from 25 μM-50 μM RepSox, most preferably such as around 50 μM RepSox. In another embodiment inhibitor of a TGF-β type I receptor comprises SD208. In another embodiment inhibitor of a TGF-β type I receptor comprises SD208, wherein the concentration of SD208 is from 0.01 μM-100 μM SD208, such as from 0.1 μM-10 μM SD208, such as from 0.1 μM-5 μM SD208, preferably such as from 0.1 μM-3 μM SD208, more preferably such as from 0.3 μM-2 μM SD208, most preferably such as around 1 μM SD208.

Other Components

The culture medium A used in the present invention is a complex serum-free medium that contains, instead of serum, a supplement of nutrients, growth factors and hormones based on a mixture of Dulbecco's modified Eagle's medium (DMEM, high glucose, no calcium, Life Technologies):Ham's F12 Nutrient Mixture (F12, Life Technologies), 3:1 v/v). In an embodiment any complex medium can be used in the present invention. In an embodiment the culture medium A described in the present invention comprises a mixture of Dulbecco's modified Eagle's medium (DMEM, high glucose, no calcium, Life Technologies):Ham's F12 Nutrient Mixture (F12, Life Technologies) in the ratio “A”:“B” by volumen. In an embodiment the ratio “A”:“B” represents any ratio. In an embodiment the ratio of “A” is higher than or the same as “B”. In another embodiment the of ratio of “A” is 1-100 and “B” is 1-100, such as wherein “A” is 1-10 and “B” is 1-100, such as wherein “A” is 2-5 and “B” is 1, such as wherein “A” is around 3 and “B” is 1.

In an embodiment the culture medium A of the present invention further comprises hydrocortisone, such as from 0.005-50 μg/ml hydrocortisone, such as from 0.05-5 μg/ml hydrocortisone, preferably such as from 0.1-2 μg/ml hydrocortisone, most preferably such as around 0.5 μg/ml hydrocortisone. In an embodiment the culture medium A of the present invention further comprises cholera toxin, such as from 0.1-1000 ng/ml cholera toxin, such as from 1-100 ng/ml cholera toxin, preferably such as from 3-20 ng/ml cholera toxin, most preferably such as around 10 ng/ml cholera toxin. In an embodiment the culture medium A of the present invention further comprises adenine, such as from 0.02-200×10⁻⁴ M adenine, such as from 0.2-20×10⁻⁴ M adenine, preferably such as from 0,5-5×10⁻⁴ M adenine, most preferably such as around 1.8×10⁻⁴ M adenine. In some embodiments, the medium A further comprises a Rho-associated coiled coil forming protein serine/threonine kinase inhibitor such as Y-27632 or a compound related thereto. Thus in an embodiment the culture medium A of the present invention further comprises Y-27632, such as from 0.1-1000 μM Y-27632, such as from 1-100 μM Y-27632, preferably such as from 3-20 μM Y-27632, most preferably such as around 10 μM Y-27632. In another embodiment the culture medium A of the present invention further comprises fetal bovine serum, such as from 0.005-50% fetal bovine serum, such as from 0.05-5 μg/ml % fetal bovine serum, preferably such as from 0.1-2% fetal bovine serum, most preferably such as around 0.5% fetal bovine serum. In an embodiment the culture medium A of the present invention further comprises epidermal growth factor, such as from 0.1-1000 ng/ml epidermal growth factor, such as from 1-100 ng/ml epidermal growth factor, preferably such as from 3-20 ng/ml epidermal growth factor, most preferably such as around 10 ng/ml epidermal growth factor. In an embodiment the culture medium A of the present invention further comprises amphiregulin, such as from 0.05-500 nM amphiregulin, such as from 0.5 50 nM amphiregulin, preferably such as from 1-10 nM amphiregulin, most preferably such as around 5 nM amphiregulin. In an embodiment the culture medium A of the present invention further comprises insulin, such as from 0.05-500 μg/ml insulin, such as from 0.5-50 μg/ml insulin, preferably such as from 1-20 μg/ml insulin, most preferably such as around 5 μg/ml insulin. In an embodiment the culture medium A of the present invention further comprises RepSox, such as from 0.5-5000 μM RepSox, such as from 5-500 μM RepSox, preferably such as from 10-100 μM RepSox, most preferably such as around 25 μM or 50 μM RepSox. In an embodiment the culture medium A of the present invention further comprises SB431542, such as from 0.1-1000 μM SB431542, such as from 1-100 μM SB431542, preferably such as from 3-20 μM SB431542, most preferably such as around 10 μM SB431542. In an embodiment the culture medium A further comprises L-glutamine, such as from 0.02-200 mM L-glutamine, preferably such as from 0.2-20 mM L-glutamine, such as from 1-10 mM L-glutamine, such as around 2 mM L-glutamine. L-glutamine may need to be replenished at regular time intervals: as is known to the skilled person, glutamine is an essential amino acid in the absence of which cells which are not capable of synthesizing it may die. Glutamine may be included in some commercial media, but due to its short shelf-life, it is customary in the art to add glutamine at regular time intervals to the culture medium. The above ranges are exemplary and the person of skill in the art knows how to adapt the glutamine concentration of the culture medium if needed.

In a most preferred embodiment the culture medium A of the present invention comprises
a mixture of Dulbecco's modified Eagle's medium (DMEM, high glucose, no calcium, Life Technologies):Ham's F12 Nutrient Mixture (F12, Life Technologies) in the ratio around 3:1 v/v; and
around 0.5 μg/ml hydrocortisone; and
around 5 μg/ml insulin; and
around 10 ng/ml cholera toxin; and
around 10 ng/ml epidermal growth factor and/or around 5 nM amphiregulin; and
around 1.8×10⁻⁴ M adenine; and
around 10 μM Y-27632; and
around 5% fetal bovine serum modified as described in the present invention; and
around 10 μM SB431542; and
around 25 μM or 50 μM RepSox; and
around 2 mM L-glutamine.

In one embodiment, the culture medium A comprises:
a mixture of Dulbecco's modified Eagle's medium (DMEM, high glucose, no calcium, Life Technologies):Ham's F12 Nutrient Mixture (F12, Life Technologies) in the ratio between 1:100 and 100:1 v/v; and
0.005-50 μg/ml hydrocortisone; and
0.05-500 μg/ml insulin; and
0.1-1000 ng/ml cholera toxin; and
0.1-1000 ng/ml epidermal growth factor and/or around 5 nM amphiregulin; and
0.02×10⁻⁴-200×10⁻⁴ M adenine; and
10 μM Y-27632; and
0.05%-500% fetal bovine serum modified as described in the present invention; and
0.1-1000 μM SB431542; and/or
0.25-250 μM or 0.50-500 μM RepSox; and
0.02-200 mM L-glutamine.

In one embodiment, the culture medium A comprises:
a mixture of Dulbecco's modified Eagle's medium (DMEM, high glucose, no calcium, Life Technologies):Ham's F12 Nutrient Mixture (F12, Life Technologies) in the ratio 3:1 v/v; and
0.5 μg/ml hydrocortisone; and
5 μg/ml insulin; and
10 ng/ml cholera toxin; and
10 ng/ml epidermal growth factor and/or around 5 nM amphiregulin; and
1.8×10⁻⁴ M adenine; and
10 μM Y-27632; and
5% fetal bovine serum modified as described in the present invention; and
10 μM SB431542; and
25 μM or 50 μM RepSox; and
2 mM L-glutamine.

In some embodiments, the culture medium A is devoid of hydrocortisone and cholera toxin. This particular embodiment is referred to as “FAD3 medium”. The FAD3 medium can be applied to tumor cell lines, including but not limited to, MCF7 or primary CD166^{high}/CD117^{low} cells or normal cells. CD166^{high}/CD117^{low} cells were found to maintain ER expression and grow better in FAD3 than in FAD2. Thus in another preferred embodiment, the culture medium A of the present invention comprises
a mixture of Dulbecco's modified Eagle's medium (DMEM, high glucose, no calcium, Life Technologies):Ham's F12 Nutrient Mixture (F12, Life Technologies) in the ratio around 3:1 v/v; and
around 5 μg/ml insulin; and
around 10 ng/ml epidermal growth factor and/or around 5 nM amphiregulin; and
around 1.8×10⁻⁴ M adenine; and
around 10 μM Y-27632; and
around 5% fetal bovine serum modified as described in the present invention; and
around 10 μM SB431542; and
around 25 μM or 50 μM RepSox; and
around 2 mM L-glutamine.

In one embodiment, the culture medium A comprises:
a mixture of Dulbecco's modified Eagle's medium (DMEM, high glucose, no calcium, Life Technologies):Ham's F12 Nutrient Mixture (F12, Life Technologies) in the ratio between 1:100 and 100:1 v/v; and
0.05-500 μg/ml insulin; and
0.1-1000 ng/ml epidermal growth factor and/or around 5 nM amphiregulin; and
0.02×10⁻⁴-200×10⁻⁴ M adenine; and
10 μM Y-27632; and
0.05%-500% fetal bovine serum modified as described in the present invention; and
0.1-1000 μM SB431542; and
0.25-250 μM or 0.50-500 μM RepSox; and
0.02-200 mM L-glutamine.

In one embodiment, the culture medium A comprises:
a mixture of Dulbecco's modified Eagle's medium (DMEM, high glucose, no calcium, Life Technologies):Ham's F12 Nutrient Mixture (F12, Life Technologies) in the ratio 3:1 v/v; and
5 μg/ml insulin; and
10 ng/ml epidermal growth factor and/or around 5 nM amphiregulin; and
1.8×10⁻⁴ M adenine; and
10 μM Y-27632; and
5% fetal bovine serum modified as described in the present invention; and
10 μM SB431542; and
25 μM or 50 μM RepSox; and
2 mM L-glutamine.

In some embodiments, the culture medium A is devoid of hydrocortisone. In some embodiments, the medium is devoid of cholera toxin.

The inventors have found that the FAD3 medium is a specific embodiment of culture medium A which is particularly advantageous, as it results in the cells maintaining a stable phenotype, i.e. cells which are capable of remaining luminal cells. The FAD3 medium in other words reduces or prevents the apparition of basal cells. The culture A described herein above are the composition used by the present inventors, however minor changes in one or more of the ingredients may still result in growth release of ER^{pos} as described by the present invention and are also envisaged.

In an embodiment the term “around” in the section above regarding ingredients of the culture medium A should be interpreted to cover a factor 10 plus or minus deviations from the concentrations described above for each individual ingredient. To illustrate this around 50 μM RepSox should be interpreted as from 5-500 μM RepSox. In another embodiment the term “around” in the section above regarding ingredients of the culture medium A should be interpreted to cover a factor 2 plus or minus deviations from the concentrations described above for each individual ingredient. To illustrate this around 50 μM RepSox should be interpreted as from 25-100 μM RepSox. In another embodiment the term “around” in the section above regarding ingredients of the culture medium A should be interpreted to cover a factor 1.5 plus or minus deviations from the concentrations described above for each individual ingredient. To illustrate this around 50 μM RepSox should be interpreted as from 33.3-75 μM RepSox. In another embodiment the term “around” in the section above regarding ingredients of the culture medium A should be interpreted to cover a factor 1.2 plus or minus deviations from the concentrations described above for each individual ingredient. To illustrate this around 50 μM RepSox should be interpreted as from 41.6-60 μM RepSox.

Culture Medium B/BBMYAB

An aspect of the present invention relates to a culture medium B, capable of generating CD326^{high}/CD271^{high} ER positive cells. These cells may be the starting point for generating cell lines, cell strains and/or cell strains with extended lifespan of the present invention. In another aspect the culture medium B as described herein is capable of generating 3-D epithelial organization normal phenotype as described herein or supports growth of cancer cells from malignant breast tumors. Culture medium B may be referred to as BBMYAB medium in the present invention.

In an embodiment the culture medium B of the present invention further comprises Y-27632, such as from 0.1-1000 μM Y-27632, such as from 1-100 μM Y-27632, preferably such as from 3-20 μM Y-27632, most preferably such as around 10 μM Y-27632. In an embodiment the culture medium B of the present invention further comprises adenine, such as from 0.02-200×10⁻⁴ M adenine, such as from 0.2-20×10⁻⁴ M adenine, preferably such as from 0,5-5×10⁻⁴ M adenine, most preferably such as around 1.8×10⁻⁴ M adenine. In an embodiment the culture medium B of the present invention further comprises serum replacement B27, such as from 0.02-300 μl/ml serum replacement B27, such as from 0.2-100 μl/ml serum replacement B27, preferably such as from 5-50 μl/ml serum replacement B27, most preferably such as around 20 μl/ml serum replacement B27. In an embodiment the culture medium B of the present invention further comprises BBM without HEPES (as described in Pasic et al 2011).

In a most preferred embodiment the culture medium B of the present invention comprises

BBM without HEPES; and
around 1.8×10⁻⁴ M adenine; and
around 10 μM Y-27632; and
around 20 μl/ml serum replacement B27; and
around 2 mM L-glutamine.

In an embodiment the culture medium B comprises L-glutamine, such as from 0.02-200 mM L-glutamine, preferably such as from 0.2-20 mM L-glutamine, such as from 1-10 mM L-glutamine, such as around 2 mM L-glutamine. L-glutamine may need to be replenished at regular time intervals: as is known to the skilled person, glutamine is an essential amino acid in the absence of which cells which are not capable of synthesizing it may die. Glutamine may be included in some commercial media, but due to its short shelf-life, it is customary in the art to add glutamine at regular time intervals to the culture medium. The above ranges are exemplary and the person of skill in the art knows how to adapt the glutamine concentration of the culture medium if needed.

The culture medium B described herein above is the composition used by the present inventors, however minor changes in one or more of the ingredients may still generate CD326^{high}/CD271^{high} ER positive cells. In an embodiment the term “around” in the section above regarding ingredients of the culture medium B should be interpreted to cover a factor 10 plus or minus deviations from the concentrations described above for each individual ingredient, as described above. In another embodiment the term “around” in the section above regarding ingredients of the culture medium B should be interpreted to cover a factor 2 plus or minus deviations from the concentrations described above for each individual ingredient, as described above. In another embodiment the term “around” in the section above regarding ingredients of the culture medium B should be interpreted to cover a factor 1.5 plus or minus deviations from the concentrations described above for each individual ingredient, as described above. In another embodiment the term “around” in the section above regarding ingredients of the culture medium B should be interpreted to cover a factor 1.2 plus or minus deviations from the concentrations described above for each individual ingredient, as described above.

Generation of (Immortalized) Cell Lines

In an embodiment the (immortalized) cell lines described by the present invention may derive from transfected primary or early passage (e.g. second, third, fourth, and /or fifth passage) cell strains. Transduction with hTERT and/or shp16 may yield immortalized cell lines (or just cell lines) upon culturing in culture medium A, described herein above, e.g. in Primaria™ flasks.

Generation of Cell Strains with Extended Lifespan

In an preferred embodiment the cell strains with extended lifespan as described herein are generated from primary cells and/or cell strains with a CD166^{high}/CD117^{low} phenotype cultured in culture medium B, described herein above, in presence of feeder cells e.g. NIH3T3 feeder cells thereby obtaining CD326^{high}/CD271^{high} ER positive cells which, when cultured in culture medium A, e.g. in Primaria™ flasks, yields cell strains with extended lifespan.

Generation of Cell Strains

In a preferred embodiment the cell strains of the present invention are cultured in culture medium A e.g. in Primaria™ flasks. Typically the cell strains of the present invention are ER positive for 1-6 passages.

A method of Distinguishing an Estrogen Receptor Positive Cell

Current methods for identifying ER^{pos} cells rely on direct detection of the ER or by detection of ER related signal pathways. However, all methods are invasive and do not allow survival of the ER^{pos} cells. Thus the current methods of identifying ER^{pos} cells do not allow collection of the cells. The present invention provides a method of distinguishing ER^{pos} and ER^{neg} cell using a non-invasive method which allows collection of the analyzed cells e.g. by FACS. Thus an aspect of the present invention relates to a method of distinguishing an estrogen receptor positive cell and an estrogen receptor negative cell based on cell surface proteins, said method comprising the steps of:
a. providing a cell as described by the present invention, and
b. determining if said cell or cell line has a Ks20.8^{high} or Ks20.8^{low} phenotype as described by the present invention,

wherein a cell or a cell line with a Ks20.8^{high} phenotype is estrogen receptor positive and a cell or cell line with a Ks20.8^{low} phenotype is estrogen receptor negative. In an embodiment the Ks20.8 phenotype is determined by an anti-cytokeratin 20 antibody, such as the clone Ks20.8 antibody. In another embodiment said estrogen receptor positive cell further has a CD326^{high}/CD271^{low} phenotype. In another embodiment said estrogen receptor positive cell further has a CD166^{high}/CD117^{low} phenotype.

The present inventors have shown that cells with a Ks20.8^{high}/CD326^{high}/CD271^{low}/CD166^{high}/CD117^{low} phenotype are estrogen receptor positive. In another embodiment cells with a CD326^{high}/CD271^{low}/CD166^{low}/CD117^{high} and Ks20.8^{high} or Ks20.8^{low} phenotype are estrogen receptor positive.

Release of Growth Restraint of an ER^{pos} Primary Breast Epithelial Cell

So far cultures of ER^{pos} cells lose expression of the estrogen receptor after only a few days (Ref 8). Furthermore, breast epithelial cells have a very limited lifespan in vitro. Solving these two issues may pave the way for development of in vitro assays with ER^{pos} cells. The present invention provides a solution for both the above mentioned issues. Thus in an aspect of the present invention relates to a method for releasing the growth restraint of a primary breast epithelial cell, while maintaining an estrogen receptor positive phenotype, said method comprising the steps of:
a. isolating a primary breast epithelial cell as described by the present invention; and
b. culturing said cell in a culture medium A as described in the present invention,

thereby releasing the growth restraint of said isolated cell, while maintaining an estrogen positive phenotype.

Present methods allow ER^{pos} breast epithelial cell strains to be cultured through approximately four-six cell passages, which may correspond to less than 15 population doublings, before the cell strains stop growing and lose their ER^{pos} phenotype. The growth restraint ER^{pos} cells has been a major challenge for the scientific community. The present invention provides methods for releasing the growth restraint of ER^{pos} cells. The present inventors have identified TGF-β type I receptor inhibitors as very important factor for releasing the growth restraint of ER^{pos} cells. The inventors have shown that inclusion of one or more TGF-β type I receptor inhibitors allow cells to remain ER^{pos} for at least 12 population doublings.

Thus an embodiment of the present invention relates to cells or a cell strain with extended lifespan which remains estrogen receptor positive for at least 15 population doublings. An embodiment of the present invention relates to cells or a cell strain with extended lifespan which remains estrogen receptor positive for at least 15 population doublings, such as at least 20 population doublings, such as at least 25 population doublings, such as at least 30 population doublings, such as at least 40 population doublings, such as at least 50 population doublings.

Inclusion of RepSox in the culture medium A as well as using murine fibroblast feeder cells as described herein further extend the time which the cells or cell strains with extended lifespan can remain ER^{pos} to at least 25 population doublings, such as at least 30 population doublings, such as at least 40 population doublings, such as at least 50 population doublings, such as at least 75 population doublings, such as at least 100 population doublings.

ER^{pos} Tumorigenic Cells and Assays

ER^{pos} cells are involved in most forms of human breast cancers. Thus it is important to study tumorigenic cells which are ER^{pos}. The present invention provides methods for producing assays for studying ER^{pos} tumorigenic cells. Thus an aspect of the present invention relates to a method for producing a tumorigenic cell, cell line and/or cell strain with extended lifespan and/or cell strain from an estrogen receptor positive cell, cell line and/or cell strain with extended lifespan and/or cell strain, the method comprising the step of:
a. providing estrogen receptor positive cell, cell line and/or cell strain with extended lifespan and/or cell strain as described by the present invention, and
b. contacting said cell to a tumorigenic agent which transforms said cell, cell line or cell strain with extended lifespan into a tumorigenic cell, cell line and/or cell strain with extended lifespan and/or cell strain capable of forming a tumor in culture or in vivo. By forming a tumor in culture is meant that the cells exhibit tumor cell characteristics in the culture.

Another aspect of the present invention relates to a tumorigenic cell, cell line and/or cell strain with extended lifespan and/or cell strain produced according to the present invention.

A further aspect of the present invention relates to a method of culturing cancer cells, tumorigenic cells and/or tumors from normal and/or luminal epithelial cells, wherein said normal and/or luminal epithelial cells are grown in a cholera toxin free medium comprising FAD2, TGFβR inhibitor, with or without estrogen or in BBMYAB on fibroblast feeders with or without estrogen as described by the present invention.

Another aspect of the present invention relates to an in vitro method for identifying an agent which reduces proliferation and/or survival of a tumorigenic cell, cell line, and/or cell strain, and/or cell strain comprising the steps of:
a. contacting a tumorigenic cell of the present invention with a candidate agent;
b. assessing the ability of said candidate agent for its ability to reduce proliferation and/or survival of said tumorigenic cell, cell line and/or cell strain with extended lifespan and/or cell strain, under condition appropriate for the candidate agent to enter said cell, cell line and/or cell strain with extended lifespan and/or cell strain;
c. determining the extent to which proliferation of the tumorigenic cell, cell line and/or cell strain with extended lifespan and/or cell strain occurs in the presence of the candidate agent; and
d. comparing the extent determined with the extent to which proliferation of the tumorigenic cell, cell line and/or cell strain with extended lifespan and/or cell strain occurs under the same conditions, but in absence of the candidate agent,
wherein if the proliferation and/or survival occurs to a lesser extent in the presence of the candidate agent than in its absence, the candidate agent is an agent which reduces proliferation and/or survival of said tumorigenic cell.

A further aspect of the present invention relates to identifying a gene the expression of which in a tumorigenic cell, cell line and/or cell strain with extended lifespan and/or cell strain is related to or involved in metastasis of such cell in vivo, the method comprising the steps of:
a. introducing a candidate gene into a tumorigenic cell described in the present invention, thereby producing a modified tumorigenic cell, cell line and/or cell strain with extended lifespan and/or cell strain;
b. introducing the modified tumorigenic cell, cell line and/or cell strain with extended lifespan and/or cell strain into a test animal;
c. maintaining said test animal under condition appropriate for metastasis to occur; and
d. determining whether metastasis of the modified tumorigenic cell, cell line and/or cell strain with extended lifespan and/or cell strain occur,
wherein if metastasis occurs, the candidate gene is a gene the expression of which in a tumorigenic cell, cell line and/or cell strain with extended lifespan and/or cell strain is related to or involved in in metastasis of such cells in vivo.

Another aspect of the present invention relates to a method of identifying a gene product which is upregulated or downregulated in a tumor cell, but not in a normal cell of the same type, the method comprising the steps of:
a. analyzing the tumorigenic cell, cell line and/or cell strain with extended lifespan and/or cell strain of the present invention;
b. analyzing a normal cell from which said tumorigenic cell, cell line and/or cell strain with extended lifespan and/or cell strain is derived from; and
c. comparing the gene products produced in step (a) with step (b), whereby a gene product which is upregulated or downregulated is identified.

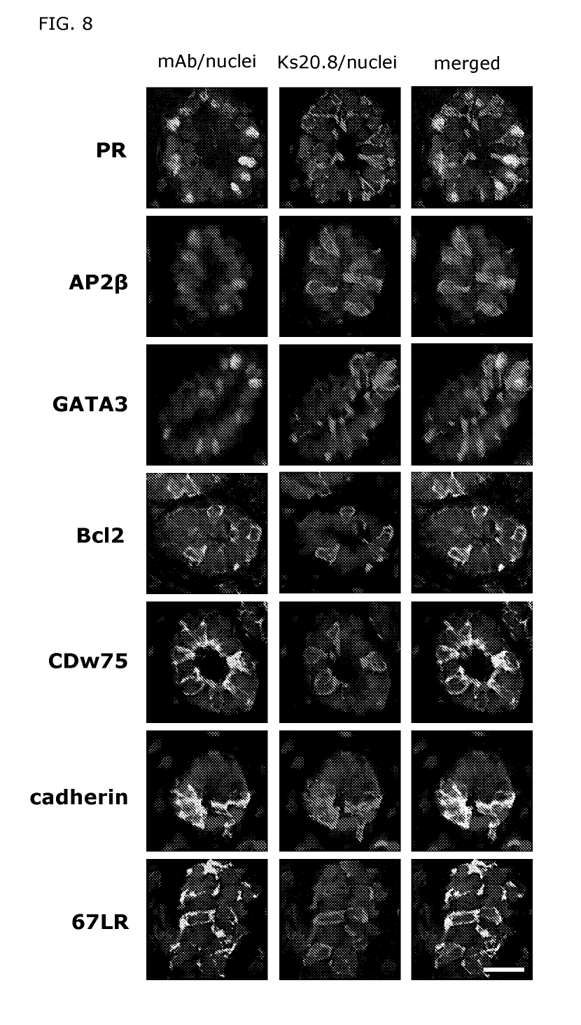

The present inventors have demonstrated that ER^{pos} human breast epithelial cells can be identified in situ by a panel of markers, including PR, AP2β, GATA3, Bcl2, CDw75, N-cadherin and 67LR (FIG. 8). Furthermore the present inventors have shown that ER^{pos} human breast epithelial cells are enriched for in the CD326^{high}/CD271^{low}/CD166^{high}/CD117^{low} gate or the CD326^{high}/CD271^{low}/CD166^{low}/CD117^{high} , and that these cells consistently and that ER^{pos} cells can be identified by can be identified by an antibody, clone Ks20.8, originally raised against keratin 20 (Moll et al., 1992). The distinction between ER^{pos} cells, other luminal cells and basal cells is further supported by their relative expression of a panel of transcripts as described herein.

The present inventors have also shown that ER^{pos} cells can be released from growth restraint and sustained by TGFβR2i and that ER^{pos} cells under these conditions can be expanded considerably. The finding of the present inventors may represent a paradigm shift in studying ER expression and function in the breast, which in the future no longer needs to rely exclusively on organoid short-term cultures, in vivo mouse models and human breast cancer cell lines. In addition, the present inventors have answered the longstanding question of whether ER^{pos} cells can self-renew and have further established that ER^{pos} can be generated from ER^{neg} progenitors, here represented by CD117^{hi} luminal cells. Surprisingly the present inventors have shown that the response to TGFβR2i and/or small molecule inhibitors of TGF-β signaling is specific to luminal cells only, as neither basal cells, fibroblasts nor ER^{neg} normal breast cell lines are able to switch on ER in response to TGFβR2i. Even though ER^{pos} HBEC are here shown to be proliferating, we refrain from referring to this population as progenitors altogether. Thus, most ER^{pos} HBEC are considered to be close to the base in the hierarchy. However, as has been hypothesized for breast cancer hierarchies, our data could be interpreted in favor of the existence of bi-directionality also in the normal breast hierarchy, i. e. upon appropriate stimuli apparently differentiated cells turn out in reality to be facultative progenitors.

Long term culturing of ER^{pos} human breast epithelial cells in the presence of TGFβR2i and/or small molecule inhibitors of TGF-β signaling is clinically relevant because it may help explain an enigmatic difference between the normal human breast and breast cancers. Thus, in the normal breast in vivo there is a strict dissociation between steroid receptor expression and proliferation (Clarke et al., 1997). In breast cancer and to a varying degree in precancerous breast lesions this negative association is lost (Shoker et al., 1999). It has been speculated that this may represent an important early change in the genesis of breast cancer either reflecting a failure to down-regulate ER as cells enter the cell cycle or a failure to suppress division of ER^{pos} cancer cells. Our data indeed are in favor of the latter possibility because ER^{pos} human breast epithelial cells do divide if TGFβ signaling is perturbed, a very likely scenario in cancer. While in primary breast cancer a normal-like TGFβ signaling is still in operation to restrain growth, in metastatic breast cancer TGFβ signaling has shifted to that of an epithelial-mesenchymal transition response.

Another implication of the culture protocol of the present invention is that it represents a much in demand cell based assay for estrogen action on normal cells. It is already well established that estrogen is a mitogen for ER-positive breast cancer. However, its role in relation to ER^{pos} human breast epithelial cells has remained a mystery due to lack of ER^{pos} cell culture models, and because ectopic expression of ER in basal cell lines has provided the paradoxical result of growth inhibition (Lundholt et al., 2001; Zajchowski et al., 1993). As a proof of principle, we here show that the TGFβR2i protocol serves as a physiologically relevant cell based assay for estrogen action, and moreover, in ER^{pos} cells, several estrogen-responsive genes downstream of ER are up-regulated. An optimal response is observed when EGF is omitted. Thus, the TGFβR2i protocol represents a cell based assay for gauging estrogen action reminiscent of its action in vivo. Intriguingly, in the presence of fibroblasts, growth of ER^{pos} human breast epithelial cells is stimulated by estrogen irrespective of the presence of EGF. This implies that stromal factors modulate ER activity. Wth the relevant representatives of ER^{pos} cells in hand, the complexity of this interaction can now be elucidated.

The inventors data further shows that TGF-β signaling is key to the CD166^{high} phenotype, and the exact mechanisms by which TGFβR2i generate ER^{pos} cells clearly await further scrutiny. It cannot be excluded, however, that what the inventors observe is part of a more general association between CD166 expression and TGF-β signaling. Thus, very recently others have found that TGF-β signaling is a main driver of the behavior of CD166-expressing prostate cells.

The data illustrate, however, that apparent post-mitotic cells in vivo, given the right conditions may multiply considerably, yet still exhibiting a definitive lifespan, and the approach moreover serves as a platform for extending the life span of ER^{pos} cells into long-term culture.

There are multiple implications of the present invention. Firstly, knowledge about how to turn on and off the ER expression in non-malignant breast epithelial cells may offer an alternative to selective estrogen receptor modulators (SERMs) in prevention of breast cancer in women with elevated risk of disease. Secondly, a reproducible source of normal human ER^{pos} Human breast epithelial cells will represent a first step towards a physiological, cell-based screen for environmental estrogenic activity and susceptibility of normal cells to breast cancer. And thirdly, the protocol may serve to test the functional role of recently identified SN Ps associated with increased risk of breast cancer. In conclusion, we have provided a cell based assay that allows normal breast ER^{pos} cells to present themselves for investigation. These findings may fuel future advances in breast cancer prevention.

### EXAMPLES

Methods

Tissue.

Normal breast biopsies were collected with consent from women undergoing reduction mammoplasty for cosmetic reasons. The use of human material has been reviewed by the Regional Scientific Ethical Committees (Region Hovedstaden) and approved with reference to H-2-2011-052. Normal breast tissue was prepared as previously described (Ronnov-Jessen et al. 1993). Upon collagenase treatment, fibroblasts and epithelial organoids were either used directly or frozen in liquid nitrogen for later use.

Fluorescence Activated Cell Sorting (FACS).

To reveal epithelial cell composition and to isolate single cells, organoids from twelve biopsies were trypsinized, filtered through a 100 μm filter and resuspended in HEPES buffer supplemented with 0.5% BSA (bovine fraction V; Sigma-Aldrich) and 2 mM EDTA (Merck), pH 7.5. The suspended cells were incubated for 45 min at 4° C. in the presence of conjugated monoclonal antibodies EpCAM/CD326-PerCP cy5.5 (9C4, 1:20, BioLegend), NGFR (neurotrophin receptor, p75)/CD271-APC (ME20.4, 1:50, Cedarlande Laboratories) to separate basal and luminal cells, and activated leukocyte cell adhesion molecule CD antigen, ALCAM/CD166-AF488 (3A6, 1:20, AbD Serotec) and C-Kit/CD117-PE (104D2,1:20, BD) to separate luminal cells into luminal K8^{lo}/ER^{neg} (CD166^{low}/CD117^{high}) cells and luminal K8^{high}/ER^{pos} (CD166^{high}/CD117^{low}cells. Upon incubation, the cells were washed twice in HEPES/BSA/EDTA buffer and filtered through a 20 μm filter cup (Filcons) to prevent clogging of the FACS apparatus. Propidium iodide (Invitrogen) was added at a concentration of 1 μg/ml, and the cells were analyzed and sorted using a flow cytometer (FACSAria; BD Biosciences). An alternative way to isolate the two luminal subpopulations is to incubate for 30 min with EpCAM/CD326-PerCP cy5.5 and ALCAM/CD166-AF488 along with 67 kDa Laminin Receptor (MLuC5 1:50, Abcam) followed by 20 min incubation with secondary antibody Alexa Fluor®647 Goat Anti-Mouse IgM (1:500, Life Technologies).

To detect TGFβ receptors on the cell surface, third passage CD166^{high}/CD117^{low} cells were incubated with monoclonal antibody TGURII (MM0056-4F14, 1:20, Abcam) followed by secondary antibody AF488 (IgG1, 1:500) and analyzed by FACS. Overlay histograms were produced using Flowing Software 2.5.1 (University of Turku, Finland).

To establish fibroblast feeders, fourth passage fibroblasts were incubated as described above with monoclonal antibodies CD26 (202-36, 1:200, Abcam) and conjugated CD105-AF488 (1:25, AbD Serotec) followed by secondary antibody AF647 (IgG2b, 1:500) to isolate CD105^{high}/CD26¹° w cells.

Cell Culture.

Upon sorting, the primary cell populations were plated in Primaria™ T-25 flasks (#3813, Becton Dickenson) in the presence of “FAD2” (Dulbecco's modified Eagle's medium (DMEM, high glucose, no calcium, Life Technologies):Ham's F12 Nutrient Mixture (F12, Life Technologies), 3:1 v/v), 0.5 μg/ml hydrocortisone, 5 μg/ml insulin, 10 ng/ml cholera toxin (Sigma Aldrich), 10 ng/ml epidermal growth factor (Peprotech), 1.8×10⁻⁴ M adenine, 10 μM Y-27632 and 5% fetal bovine serum (Sigma Aldrich), modified from Liu et al. 2012 and Tan et al. 2013). Upon plating, which could take up to two days for the luminal K8^{lo}/ER^{neg} (CD166^{low}/CD117^{high}) cells and thus determined the time point for addition of TGFβR2i, a combination of the selective inhibitor of TGF-β type I receptor activin receptor-like kinase ALKS, ALK4 and ALK7, SB431542 Laping et al. 2002 (10 μM, S4317, Sigma Aldrich) and an inhibitor of autophosphorylation of ALK-5, RepSox (Gellibert et al. 2004 and Ichida et al. 2009) (25 or 50 μM, R0158, Sigma Aldrich) was added. To test the specificity of the effect of TGFβR2i, in some experiments SB431542 alone or the double concentration of SB431542 was used instead of TGFβR2i or RepSox was replaced by another TGF-β type I receptor activin receptor-like kinase ALKS inhibitor, SD208 (1 μM, Tocris Bioscience). The vehicle, dimethyl sulfoxide (Sigma Aldrich), was included in all experiments added in in appropriate concentrations for control cultures. Gentamycin (50 μg/ml, Biological Industries) was added to the cultures for one week after sorting, otherwise antibiotics were not included. To address whether propagation of ER^{pos} cells was restricted to the FAD2 medium, CD166^{high}/CD117^{low} cells sorted from one of the biopsies were plated at a density of 4000 cells/cm² on Primaria™ 6-well in either FAD2, M87A (Garbe et al. 2009), MEGM (LONZA) or WIT-P-NC™ (STEMGENT) medium including cholera toxin (100 ng/ml) (Ince et al. 2007) and the cultures were observed for plating for 3 days. The possible induction of ER by TGFβR2i in other breast cells was further tested in MCF-10A (Soule et al. 1990), HMT-3522 (Briand et al. 1987), fibroblasts purified from normal breast tissue (Ronnov-Jessen et al. 1993), CD117^{high} cells purified from three different biopsies, as well as in basal cells isolated by FACS as described above and cultured on irradiated NIH-3T3 feeders (Liu et al. 2012) prior to exposure to TGFβR2i for up to seven days followed by staining for ER and Ks.20.8. Fibroblasts were routinely grown to confluency on collagen coated T25 flasks (Nunc, 8 μg collagen/cm², PureColl, CellSystems) in DMEM/F12 (Life Technologies, cat. no. 21041), with 2 mM glutamine and 5% FBS prior to co-culture with CD166^{high}/CD117^{low} luminal cells.

Cloning efficiency at low density in FAD2 was observed by plating the sorted basal cells, and the luminal populations CD166^{low}/CD117^{high} and CD166^{high}/CD117^{low} luminal cells, respectively, at 400 cells/cm² and culturing for 14 days, followed by fixation in methanol for 5 min at −20° C. and counterstaining of nuclei with hematoxylin. To assess the Ks20.8 expression of the sorted populations and to further assess whether starting the culture at a higher density would change the expression, cells were seeded at 3000 cells/cm² and cultured for 9 days prior to immunocytochemical staining.

To quantify the frequency of Ks20.8^{high}/ER^{pos} colony forming units CD166^{high}/CD117^{low} luminal cells from three different biopsies were plated at a density of 4000 cells/cm² with or without TGFβR2i and grown for 13 days prior to staining for ER and Ks20.8 followed by quantification using an ocular grid. The number of stained colonies in three areas of each culture relative to the initial number of seeded cells was calculated.

For growth experiments cultures initially seeded at 6400 cells/cm² and grown for fifteen days in primary culture in TGFβR2i or with SB431542 alone. Next, the cultures were trypsinized and seeded at 4000 cells/cm² in triplicate cultures, and subsequently passaged at the same density before the cultures reached confluency. Parallel cultures were stained to assess Ks20.8, ER and PR expression status. The number of cells was quantified manually using a counting chamber. For extended cultivation sorted luminal Ks20.8^{high}/ER^{pos} (CD166^{high}/CD117^{low}) cells were first allowed to form colonies on irradiated NIH-3T3 feeders in BBM without HEPES (Pasic et al. 2011) with the addition of Y-27632, adenine and the serum replacement B27 (20 μl/ml, Life Technologies), and subsequently sorted by FACS upon incubation with CD326 and CD271 as described above to isolate CD326^{high}/CD271^{high} cells prior to plating and passaging in TGFβR2i culture. Population doublings were calculated as n=3.32(log UCY—log 1)+X, where n=population doubling, UCY=cell yield, 1=inoculum number, X=population doubling rate of inoculum. To determine whether continuous TGFβR2i culture was needed to sustain ER and K8 expression, cells in the sixth passage were switched to FAD2 and the number of ER^{pos} cells (n=3×100) was compared to parallel TGFβR2i cultures at day three and five.

As an alternative approach to extend the life span of Ks20.8^{high}/ER^{pos} cells, the method by Kiyono et al. (Kiyono et al., 1998) was adapted by introducing human telomerase (hTERT) and shRNA targeting P16 (shp16) to second passage CD166^{high}/CD117^{low} cells. The viral constructs, pLENTi X2 Hygro/shp16 (w192-1, AddGene #22264, a gift from Eric Campeau) and pBABE-neo-hTERT (Counter et al. 1998) (AddGene #1774, a gift from Robert Weinberg) were prepared as follows: Lentiviral particles containing the shp16 construct were generated by transient co-transfection into HEK-293T cells using the calcium phosphate method with the vesicular stomatitis virus glycoprotein (VSVG) expressing construct pCMV-VSVG as well as pol-gag construct pLP1 and rev construct pLP2. Retroviral hTERT particles were generated by transient co-transfection of the vector construct into a pantropic retroviral packaging cell line GP2-293 (Clonetech) which stably expresses the retroviral gag-pol genes. Medium was changed ˜16 hours after transfection and viral medium was collected ˜48 hours later and stored at 4° C. for further purification. High-titer stocks of the virus were purified using a 20% sucrose gradient during ultracentrifugation with a Beckman SW32Ti rotor at 25,000 rpm for 1.5 hours. Purified viral solution was stored at −80° C.

Prior to transduction, the cells were first treated with 200 mU/mIneuraminidase (N7885, Sigma) for 2 hours at 4° C., and then transduced using an high viral titer containing the pBABE-neo-hTERT construct in TGFβR2i without FBS and incubated at 37° C. in 5% CO₂ overnight. The culture medium was changed and the transduced cells underwent antibiotic selection for ten days with 500 μg/ml G418 (Life Technologies). Upon confluency, the cells were passaged and underwent an identical transduction procedure with viral particles containing the pLenti X2 Hygro/shp16 construct and subsequent antibiotic selection with 100 μg/ml hygromycin (Sigma) for more than two weeks.

To test the proliferative response to estrogen, ER^{pos} cells in TGFβR2i culture were seeded in second passage with 4000 cells/cm² in TGFβR2i with 25 μM RepSox and without EGF, supplemented with estrogen (10⁻⁸ M, β-estradiol, E2758, Sigma-Aldrich) and cultured for up to 13 days with medium change every other day. A primary culture from another biopsy (P959) was cultured 27 days in the presence of estrogen and without EGF before splitting. The cells were passaged at 4000 cells/cm² in triplicate cultures with estrogen or vehicle (96% ethanol). At day four the cultures were trypsinized and quantified using a counting chamber.

To assess whether the response of ER^{pos} cells to estrogen was modulated by the presence of fibroblasts, second or third passage ER^{pos} cells, with or without omission of EGF in the previous passage, were plated at a density of 5600 cells/cm² on confluent fibroblast feeders in triplicate culture. The following day, the culture medium was switched to TGFβR2i with estrogen or vehicle. Under these conditions, fibroblasts did not grow. At day eight, the cultures were trypsinized and the total cell number was quantified. Whether a continuous lack of EGF influenced the response to estrogen was tested in a similar set-up, where EGF was omitted from the medium during the entire experimental period.

For phase contrast microscopy a Nikon Diaphot 300 microscope was used.

RNA Extraction and qRT-PCR.

Total RNAs from sorted normal primary cells from eight biopsies were extracted using Trizol (Invitrogen) and were reversely transcribed using the High Capacity RNA-to-cDNA Kit (Applied Biosystems). For general gene expression profiling 2 ng of total cDNA was used to perform quantitative real time PCR using the TaqMan® Gene Expression Assays (Applied Biosystems) on CFX384 Touch™ Real-Time PCR Detection System (Bio-Rad). The primers are listed in Table 3, and real time PCR conditions were the following: 50° C. for 2 min and 95° C. for 10 min, followed by 40 cycles at 95° C. for 10 sec and 60° C. for 30 sec.

TABLE 3
List of primers used for qRT-PCR:

| Gene symbol | Assay ID | Category |
|---|---|---|
| ACTA2 | Hs00426835_gl | Basal marker |
| CD200 | Hs01033303_ml | Basal marker |
| CD271 | Hs00609977_ml | Basal marker |
| IGFBP3 | Hs00365742_gl | Basal marker |
| KRT14 | Hs00265033_ml | Basal marker |
| KRT5 | Hs00361185_ml | Basal marker |
| MME | Hs00153510_ml | Basal marker |
| NGR1 | Hs00247620_ml | Basal marker |
| SNAI2/SLUG | Hs00950344_ml | Basal marker |
| ST3GAL2 | Hs00199480_ml | Basal marker |
| AREG | Hs00950669_ml | Luminal marker |
| BIM | HS00708019_sl | Luminal marker |
| CD166 | Hs00977640_ml | Luminal marker |
| CDKN1B/P27 | Hs00153277_ml | Luminal marker |
| ESR1 | Hs00174860_ml | Luminal marker |
| FOXA1 | Hs00270129ml | Luminal marker |
| GATA3 | Hs00231122_ml | Luminal marker |
| GREB1 | Hs00536409_ml | Luminal marker |
| ID2 | Hs04187239_ml | Luminal marker |
| KRT18 | Hs02827483_gl | Luminal marker |
| KRT19 | Hs00761767_sl | Luminal marker |
| KRT8 | Hs01595539_gl | Luminal marker |
| MYB | Hs00920556_ml | Luminal marker |
| NGRN | Hs04185079_gl | Luminal marker |
| PGR | Hs01556702_ml | Luminal marker |
| SORT1 | Hs00361760_ml | Luminal marker |
| TFF1 | Hs00907239_ml | Luminal marker |
| TGM2 | Hs00190278_ml | Luminal marker |
| WNT4 | Hs01573504_ml | Luminal marker |
| DPP4 | HS00175210_ml | Luminal progenitor marker |
| ALDH1A3 | Hs00167476_ml | Luminal progenitor marker |
| CD14 | Hs00169122_gl | Luminal progenitor marker |
| CYP24A1 | Hs00167999_ml | Luminal progenitor marker |
| DAPP1 | Hs00183937_ml | Luminal progenitor marker |
| ELF5 | Hs01063022_ml | Luminal progenitor marker |
| KIT | Hs00174029_ml | Luminal progenitor marker |
| KRT15 | Hs00267035_ml | Luminal progenitor marker |
| NCALD | Hs00230737_ml | Luminal progenitor marker |
| PIGR | Hs00922561_ml | Luminal progenitor marker |
| Sox9 | Hs01001343_gl | Luminal progenitor marker |
| GAPDH | Hs02758991_gl | Reference |
| HPRT1 | Hs99999909_ml | Reference |
| TBP | Hs00427621_ml | Reference |
| TFRC | Hs00951083_ml | Reference |

In order to quantify cDNA concentration represented by cycle threshold (Ct) values, Cts were determined at the initial period of exponential amplification in triplicates and the different PCR runs were adjusted by inter-run calibrators (Bio-Rad CFX manager 3.0). Each gene expression level was to the mean of four reference gene expressions (GAPDH, HPRT1, TBP, and TFRC). The qRT-PCR data were then visualized as a heatmap using the statistical programi R or as a bar graph using the values calculated by the 2^{-ΔΔCt} method (Livak et al., 2001). For qPCR of ER, K8, FOXA1, ELF5 and K18 second passage cultures of CD166^{high} cells at a density of 8000 cells/cm² in FAD2, SB431542, or SB431542 switched to TGFβR2i culture conditions for five days prior to RNA extraction were used.

The expression of ER signaling and its downstream target genes were analyzed in CD166^{high} cells with or without TGFβR2i by using a RT² Profiler PCR array (Human estrogen signaling, Qiagen) according to manufacturer's instructions. Total 4 mg of RNA was used per array, which was performed in duplicates from two different biopsies. Three sets of E2 or ethanol (vehicle) treated second or third passage ER^{pos} cells or hTERT/shp16 transduced cells were further analyzed in triplicate for PGR and GREB1 expression with the TaqMane Gene Expression Assays in the same condition mentioned above, using 20 ng of cDNA in each PCR reaction.

Western Blotting.

Whole cell lysates were prepared for extraction by incubation in RIPA lysis buffer for 30 min at 4° C. with protease inhibitor cocktail (P8340, Sigma) and phosphatase inhibitor cocktail (Sigma, P5726). Protein were separated by a 4˜12% Novex® Bis-Tris pre-cast polyacrylamide gradient gel (Life technologies) and transferred to a PVDF membrane using iBlot® dry blotting system (Life technologies). Molecular weight was indicated by using a pre-stained protein ladder (SM0671, Fermentas). After blocking 1 hour in room temperature, the membrane was incubated with primary antibodies recognizing Smad2/3 (1:1000, #3102, Cell Signaling), pSmad 2 (1:1000, #3101, cell signaling), β-actin (1:5000, A-5441, Sigma), or ER (1:500, NCL-ER-6F11/2, Novocastra) in blocking solution with gentle rocking overnight at 4° C. Secondary antibodies conjugated with horseradish peroxidase (DAKO) were incubated for one hour at room temperature. Western blots were visualized using enhanced chemiluminescence solution (PerceECL 32106, Thermo Scientific) and a chemi-luminescence imager (Amersham Image 600, GE Healthcare life sciences).

Immunohistochemistry and Cytochemistry.

Cryostat sections, smears of sorted cells and monolayer cultures were prepared and stained by immunoperoxidase or immunofluorescence as previously described (Petersen et al. 1988, Villadsen et al. 2007, Rønnov-Jessen et al. 1992). Cellular smears for ER staining were fixed in 10% formalin for 10 min, followed by 5 min in ice-cold methanol:acetone (1:1), and incubated over night with anti-ER (Sp1, 1:10) at 4° C., followed by incubation for 30 min with Alexa Fluor 568 goat-anti rabbit IgG (Invitrogen). Antibodies are listed in Table 1. Many antibodies stain independently of fixation procedure, but of note, to stain for ER and PR, cultures were rinsed in phosphate buffered saline (PBS), pH 7.4, or sections were air dried prior to fixation for 5 min at RT in 3.7% formaldehyde, two rinses in PBS, fixation in methanol:acetone 1:1 for 5 min at −20° C., two rinses in PBS, permeabilization in 0.1% Triton X-100 in PBS, twice for 7 min, rinse in PBS and kept wet prior to application of UltraV Block (Thermo Scientific). To stain for K15, UltraV Block was substituted for 10% normal goat serum in PBS. Immunofluorescence and peroxidase stainings were evaluated, quantified and photographed using a laser-scanning microscope (LSM 510; Carl Zeiss Microlmaging, Inc.) and brightfield microscopes (Laborlux S or DM5500B, Leica), respectively. For quantification of ER, K8, K19 and p63, nuclei were counterstained with hematoxylin and counted in randomly selected fields using an ocular grid and given as the percentage of stained cells of a total of 1000 cells evaluated with a 25 times objective.

Generation of Single Cell Clones. These CD326^{high}/CD271^{low}/CD166^{high}/CD117^{low} cells transduced with the hTERT and shP16 constructs were FACS single cell cloned by FACS in passage 4 and 5 to generate a number of cell lines.

Screening Assay for Normal Morphogenic Behavior

Primary or early passage sorted luminal cells or the immortalized ER-expressing cell line are plated on a confluent feeder layer of normal breast fibroblasts, such as CD105^{high} cells (seeded at 5.6×10³ cells/cm²and cultured in DMEM/F12 with 5% FBS for 5 days prior to co-culture) in BBM without HEPES (DM EM/F-12 (Dulbecco's Modified Eagle Medium/Nutrient Mixture F-12, Life Technologies), 1 μg/ml hydrocortisone (Sigma-Aldrich), 9 μg/ml insulin (Sigma-Aldrich), 5 μg/ml Transferrin (Sigma-Aldrich), 5.2 ng/ml Na-Selenite (BD Industries), 1×10⁻² M ethanolamine (Sigma-Aldrich), 20 ng/ml basic fibroblast growth factor (bFGF) (PeproTech), 5 nM amphiregulin (R&D Systems), (Pasic, L., et al. Sustained activation of the HER1-ERK1/2-RSK signaling pathway controls myoepithelial cell fate in human mammary tissue. Genes & Dev 25, 1641-1653 (2011)) with the addition of 1.8×10⁻² M adenine (Sigma-Aldrich), 10 μM Y-27632 (Sigma-Aldrich) and 20 μl/ml B27 (Life Technologies), here called BBMYAB with or without TGFβR2i and with or without E2 (10⁻⁸ M).

Within two weeks luminal cells grow up from single cells to form polarized structures, either comprising luminal cells only or luminal cells surrounded by a layer of myoepithelial cells, reminiscent of their organization in vivo.

In this assay, we see a clear response to estrogen, i.e. presence of estrogen leads to down-regulation of its receptor and induction of the downstream progesterone receptor. In addition, larger epithelial colonied are formed in the presence of estrogen.

We envision that the assay can be used to test the effect estrogen receptor-modulating drugs in normal cells as well as in cancer cells.

Results

Identification and Isolation of Normal ER^{pos} Human Breast Epithelial Cells.

To unequivocally track ER^{pos} human breast epithelial cells at the single cell level we screened our antibody library for sensitive markers with a long half-life, e.g. cytokeratins, in culture. In situ staining of more than thirty reduction mammoplasties revealed a surprising pattern with a monoclonal antibody (clone Ks.20.8) originally raised against cytokeratin 20, a simple epithelial cytokeratin with a very restricted expression pattern and not expressed in normal human breast cells. The lack of cytokeratin 20 expression in normal breast was here confirmed with two other antibodies (listed in Table 1):

TABLE 1
List of antibodies used for immunostaining and/or FACS analysis

| Antibody | Clone | Company/Cat. No. | Peroxidase | Fluorescence | FACS |
|---|---|---|---|---|---|
| AP2β | — | Santa Cruz, sc-8976 | — | 1:50 | — |
| BCL2 | 124 | Dako, M0887 | — | 1:25 | — |
| CDw75 | LN1 | NeoMarkers, MS-130-P | — | 1:10 | — |
| CD117 | 104D2 | Dako, M7140 | — | 1:50 | — |
| CD117, PE | 104D2 | BD Biosciences, 332785 | — | — | 1:20 |
| CD166 | 3A6 | BioLegend, 343902 | — | 1:50 | — |
| CD166, Alexa | 3A6 | AbD Serotec, | — | — | 1:20 |
| Fluor 488 | | MCA1926A488 | | | |
| CD271, APC | ME20.4 | Cedarlane, CL10013APC | — | — | 1:50 |
| CD326, | 9C4 | BioLegend, 324214 | — | — | 1:10-1:20 |
| PerCP/Cy5.5 | | | | | |
| TGFβRII | MM0056- | Abcam, ab78419 | — | — | 1:20 |
| | 4F14 | | | | |
| ERα | Sp1 | Labvision, RM-9101-S | 1:25 | 1:10 | — |
| ERα | SP1 | Labvision, RM-9101-R7 | undiluted | — | — |
| ERα | 1D5 | Dako, M7047 | 1:100 | 1:25 | — |
| GATA3 | HG3-31 | Santa Cruz, sc-268 | — | 1:25 | |
| Keratin 8 | M20 | Abcam, ab9023 | 1:50 | 1:50 | — |
| Keratin 8 | TS1 | Novocastra, NCL-CK8- | 1:50 | — | — |
| | | TS1 | | | |
| Keratin 8*/K20 | Ks20.8 | Dako, M7019 | 1:25 | 1:10 | — |
| Keratin 8*/K20 | Ks20.8 | Dako, IR777 | undiluted | — | — |
| Keratin 20 | IT-Ks10-10 | Progen Biotechnik, | 1:25 | — | — |
| | | 61054 | | | |
| Keratin 20 | CK205 | Novocastra, NCL-CK20- | 1:50 | — | — |
| | | 543 | | | |
| Keratin 8/18 | NCL-5D3 | Abcam, ab90102 | — | 1:50 | — |
| Keratin 14 | LL002 | NeoMarkers, MS-115-P | 1:300 | 1:25 | — |
| Keratin 15 | LHK15 | NeoMarkers, MS-1068-P | — | 1:25 | |
| Keratin 18 | M9 | Monosan, MON3006 | 1:100 | — | |
| N-cadherin | 32 | BD Transduction | — | 1:25 | — |
| | | Laboratories, 610920 | | | |
| PR | SAN27 | Vector Laboratories, VP- | 1:100 | 1:25 | — |
| | | P987 | | | |
| 67 kDa Laminin | MLuC5 | Abcam, ab3099 | — | 1:50 | 1:50 |
| Receptor | | | | | |
| p63 | 7JUL | Novacastra (NCL-L-P63) | 1:25 | | |
| | | Vector Laboratories | | | |
| | | (VP-P960) | | | |

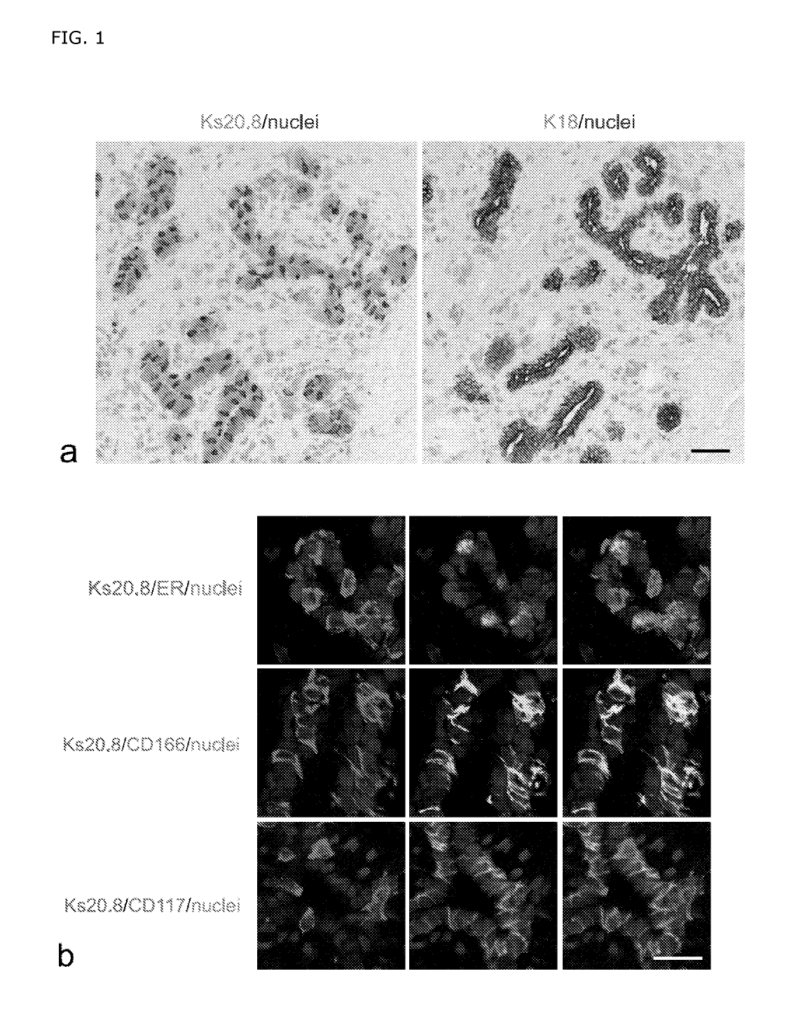

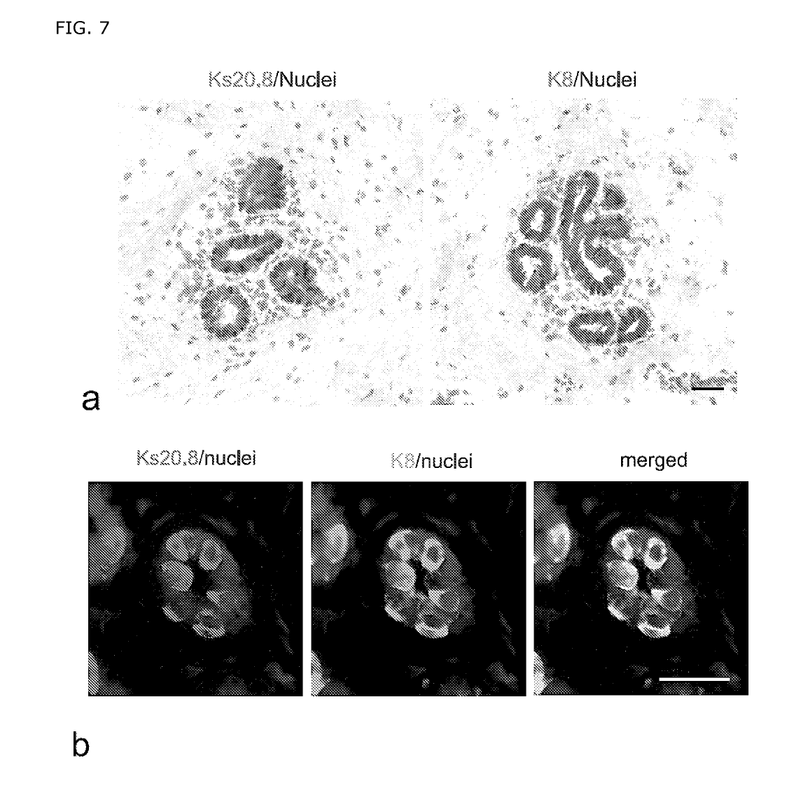

Instead, Ks20.8 stained a subpopulation of luminal cells in a unique scattered pattern (FIG. 1a). Instead, Ks20.8 stained a subpopulation of luminal cells in a unique scattered pattern (FIG. 1a). A similar pattern, albeit not as clear, was obtained with another antibody directed specifically against K20's type II partner cytokeratin K8 suggesting to indicate that Ks20.8 staining may reflect cross reactivity with this antigen (FIG. 7). Most importantly, however multicolor imaging revealed that the scattered staining with Ks20.8 very accurately reflected the presence of ER^{pos} human breast epithelial cells (FIG. 1b). The antibody screen unraveled a number of additional markers of ER^{pos} human breast epithelial cells which if applied to the multicolor imaging protocol served to substantiate an elaborate differentiation program including markers of endocrine cells such as the progesterone receptor (PR) and Activating Enhancer Binding Protein 2 Beta (AP2β), a marker of luminal differentiation, GATA3, a marker of cell survival/longevity, BcI2 , and two TGF-β-mediated, epithelial-mesenchymal transition-related markers, N-glycan/CDw75 antigen and N-cadherin, as well as the stem cell markers, ALCAM/CD166 (FIG. 1b) and the laminin receptor, 67LR (FIG. 8). Most notably, the widespread cytoplasmic Ks20.8 staining facilitated a clear distinction between ER^{pos} human breast epithelial cells and c-kit/CD117 positive progenitors (FIG. 1b). We therefore reasoned that K8^{high}/ER^{pos} human breast epithelial cells could be purified by FACS from the remainder of the luminal epithelial lineage by the cell surface markers CD166 and CD117, respectively.

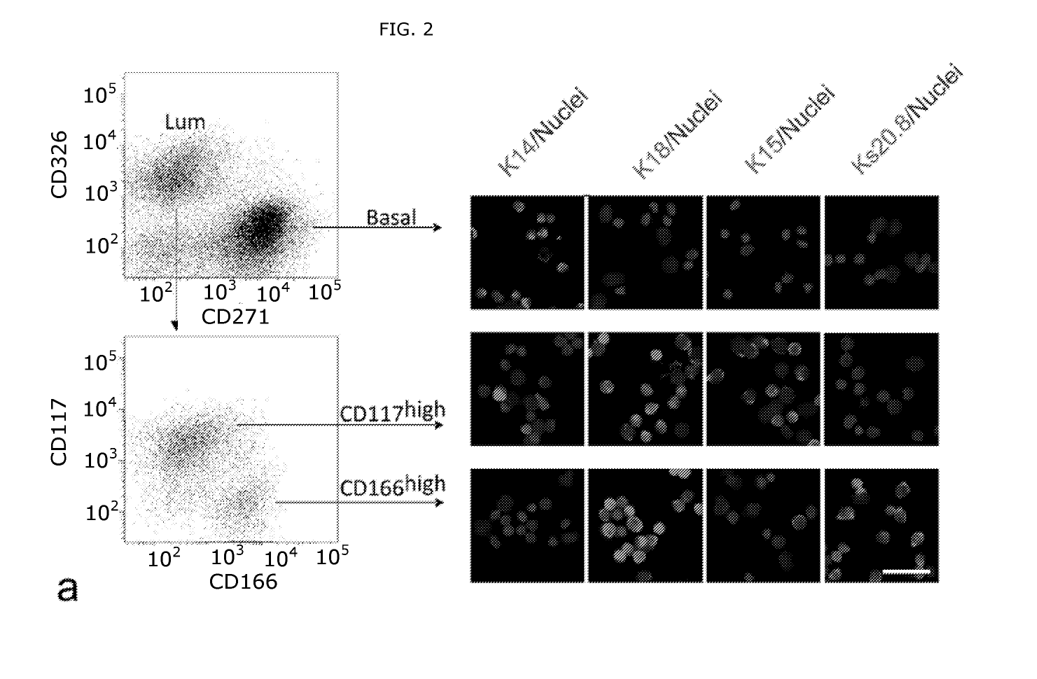

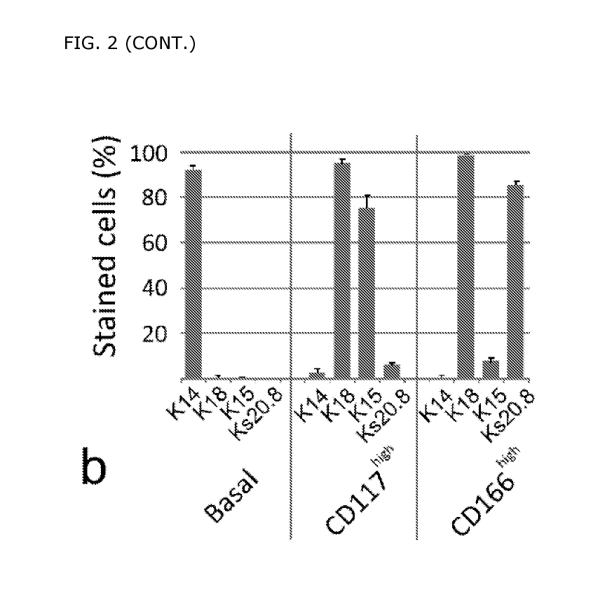

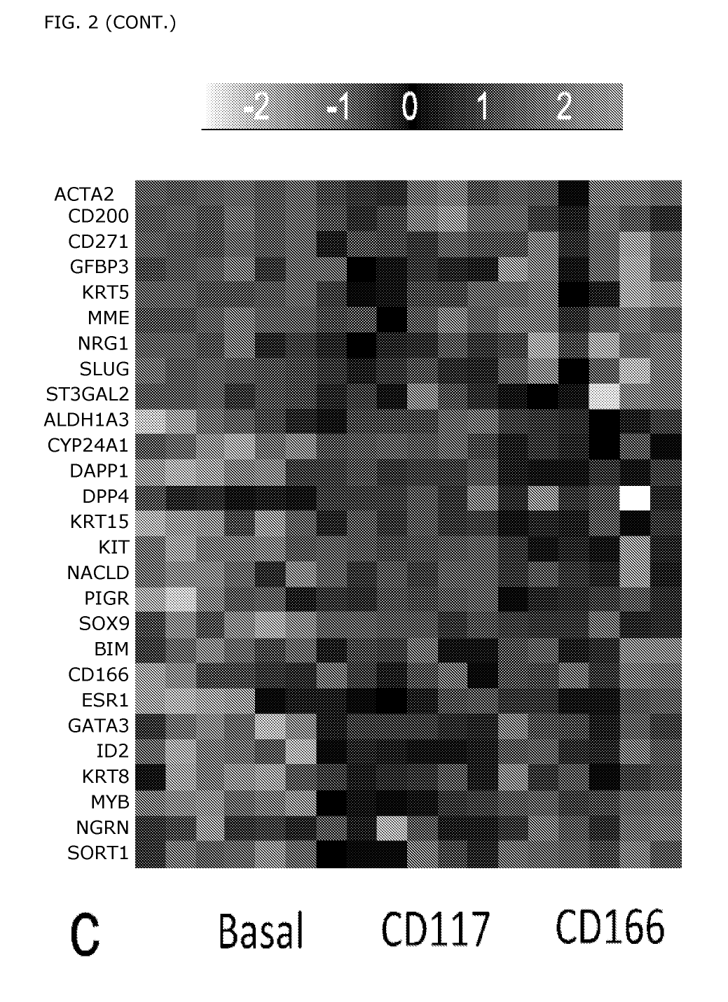

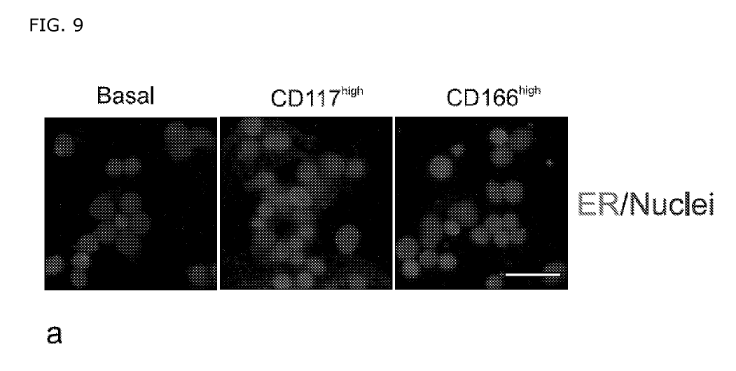

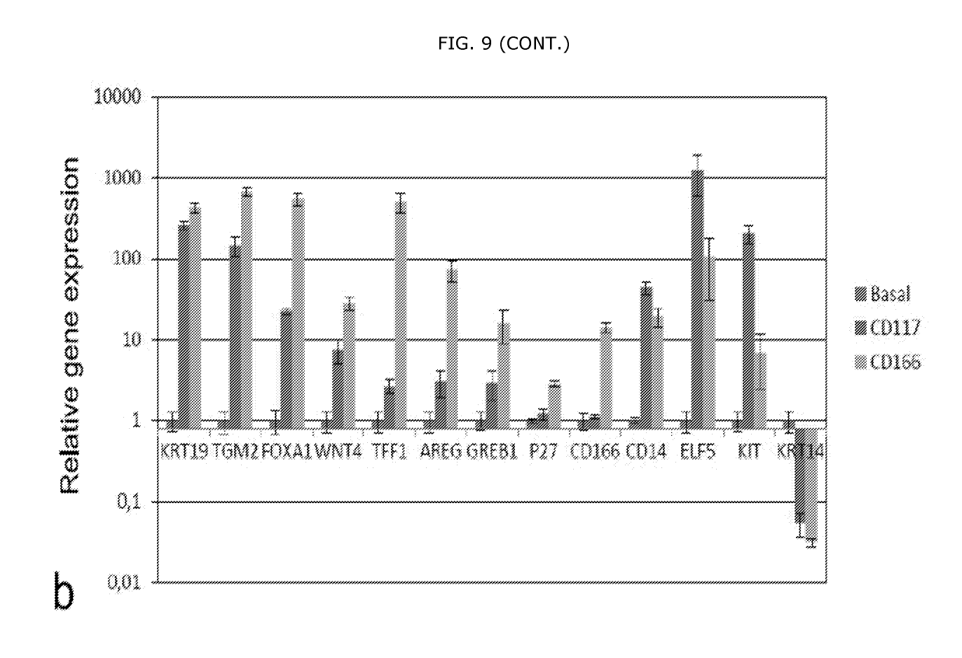

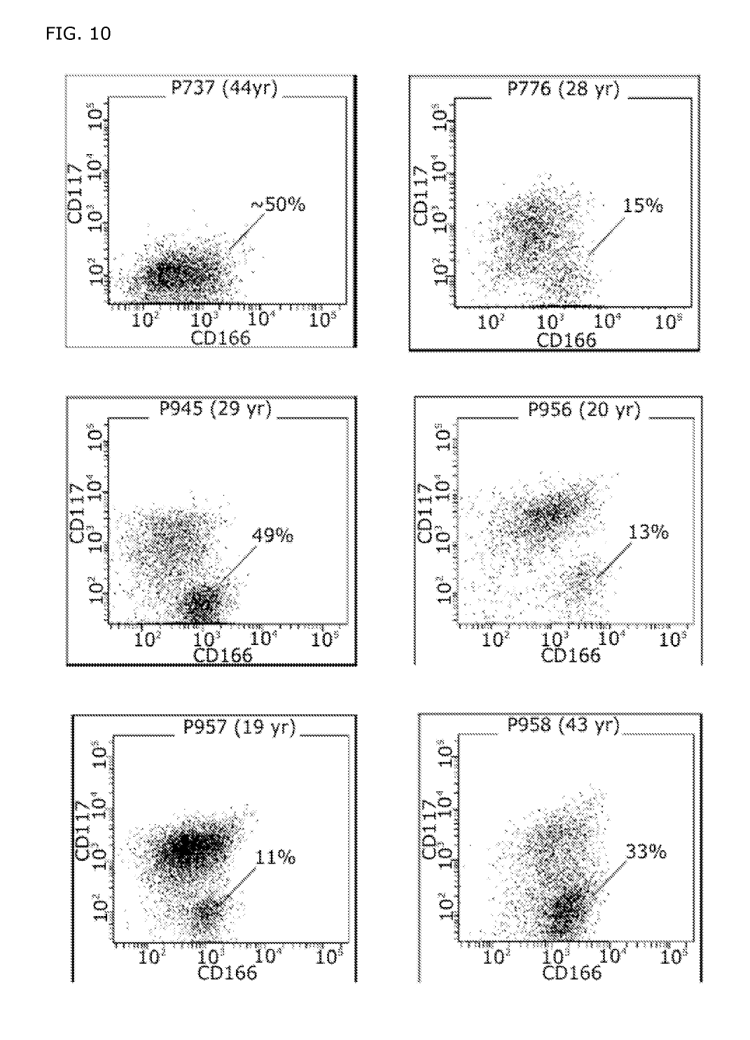

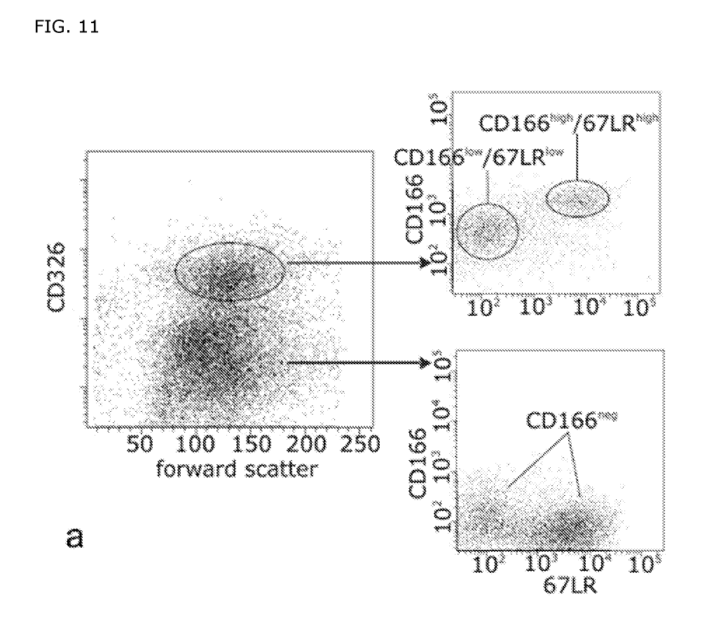

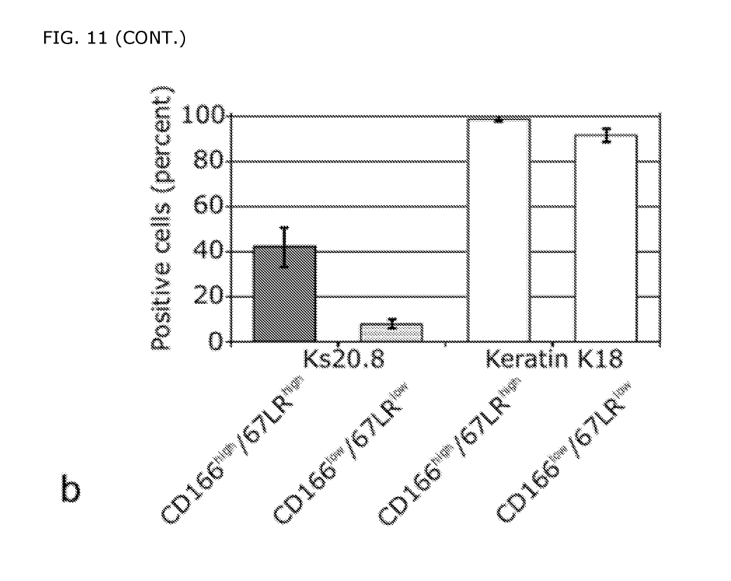

We therefore designed a FACS protocol to first separate the basal cell population from the luminal cell population based on EpCAM/CD326 and NGFR/CD271 followed by a sorting with CD166 and CD117 to further dissect the luminal compartment (FIG. 2a). This protocol yields three populations, the purity of which was assessed by staining smears with lineage and progenitor markers K14, K18, and K15 (Villadsen et al., 2007), as well as the novel ER^{pos} cell marker, Ks20.8 (FIG. 2a). In general, we found that ER^{pos} human breast epithelial cells were highly enriched in the CD166^{high}/CD117^{low} gate (FIGS. 2b and 9a). This separation of the three subpopulations was further validated by qRT-PCR (FIG. 2c), and an additional panel of markers further distinguished the two luminal subpopulations from the basal cell population (FIG. 9b). Importantly, we found that known ER signaling related genes, such as trefoil factor family-1, TFF1 (Rio et al. 1990) and growth regulation by estrogen in breast cancer 1, GREB1 (Ghosh et al. 2000) were highly expressed in CD166^{high} cells as compared to other HBEC (FIG. 9b). The degree of separation in the CD166/CD117 FACS analysis was, however, somewhat biopsy dependent. In a series of six biopsies originating from women between 19 and 44 years old, five exhibited a similar separation with 11-49% of the cells being CD166^{high}/CD117^{low}, while one biopsy apparently did not contain a CD117^{high} population (FIG. 10). As an alternative, CD117 could be replaced with the laminin receptor, 67LR, in the CD166 FACS to obtain enriched ER^{pos} human breast epithelial cells (80% increase in Ks20.8-positive cells in the 67LR^{high} gate versus the LN67low gate; (FIG. 11). In conclusion, ER^{pos} human breast epithelial cells can be isolated and traced from most normal human breast tissue samples.

ER is Lost and Growth is Restrained in CD166^{high} Derived Luminal Cells Under Conditions That Otherwise Favour Luminal Colony Formation.

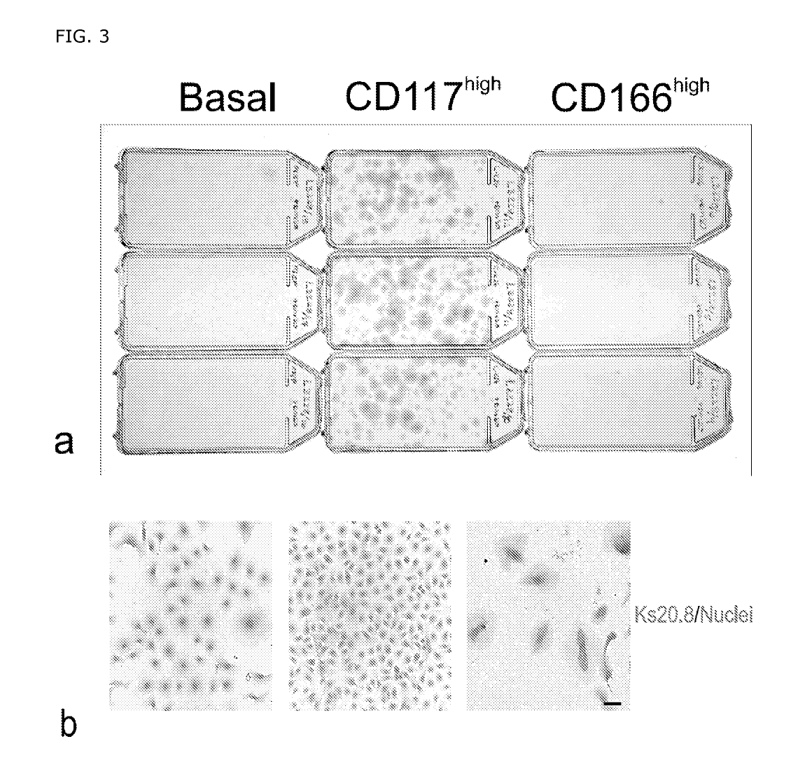

From the point of view that favourable conditions for luminal epithelial cells also apply to ER^{pos} human breast epithelial cells, we refined a protocol to permit ample colony formation of luminal cells at clonal density. As cell culture plastic we used Primaria™, a substrate with a high content of nitrogen previously shown to promote adhesion of breast cells (Rønnov-Jessen et al., 1993). The growth medium “FAD2” was modified from previously described media for culturing keratinocytes or breast epithelium on mouse fibroblast feeders (Liu, X., et al. ROCK inhibitor and feeder cells induce the conditional reprogramming of epithelial cells. Am J Pathol 180, 599-607 (2012), and Tan, D. W. M., et al. Single-cell gene expression profiling reveals funcional heterogeneity of undifferentiated human epidermal cells. Development 140, 1433-1444 (2013)). The basal medium is Ham's F12 : DMEM 1:3 similar to that of Tan et al., but with less serum, i. e. 5%, as in Liu et al. Under these conditions, we gauged for colony formation among the three FACS gated populations described above. When plated at a clonal density of 400 cells/cm², indeed colony forming luminal cells from the CD117^{high} gate were highly favoured over basal cells (FIG. 3a). However, it was also clear that the ER^{pos} human breast epithelial cells from the CD166^{high} gate entirely failed to form colonies under similar conditions. Moreover, while CD166^{high} cells plated and survived well, they completely lost ER. However, tracing with Ks20.8 revealed that the cells indeed represented ER^{pos} human breast epithelial cells—albeit essentially quiescent and without ER (FIG. 3b). By comparison, other HBEC culture media, i. e. M87A, MEGM or WIT-P-NC™ did not support plating of CD166^{high} cells. Therefore, we conclude that failure of culturing ER^{pos} human breast epithelial cells is caused by both lack of growth and loss of ER expression under conditions otherwise favouring propagation of luminal epithelial cells.

Small Molecule Inhibitors of TGF-β Signaling Induce ER Expression and Liberate ER^{pos} Human Breast Epithelial Cells from Growth Restraint.

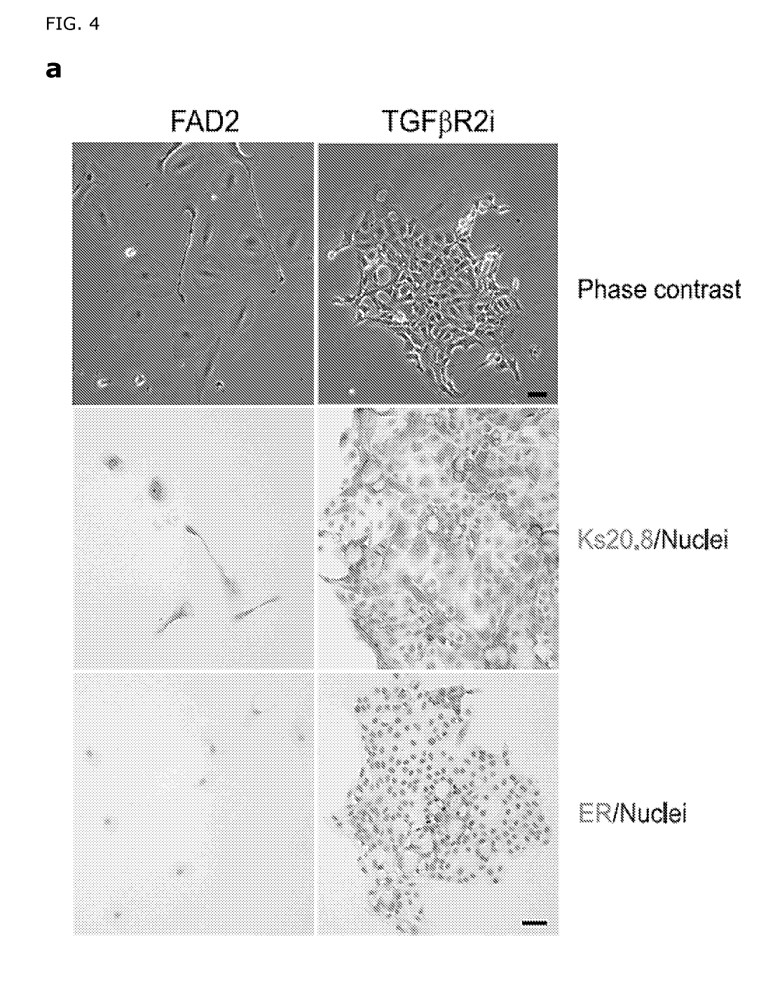

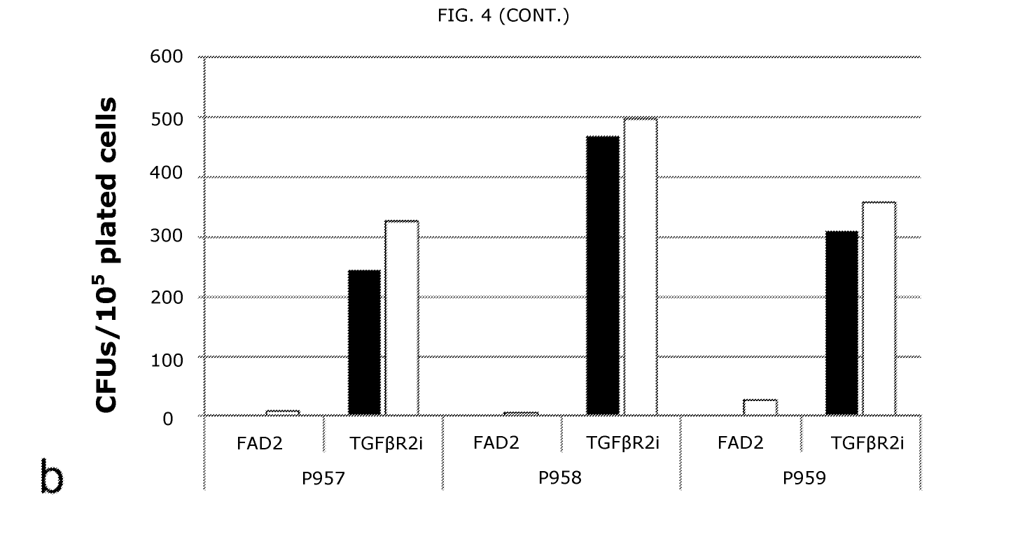

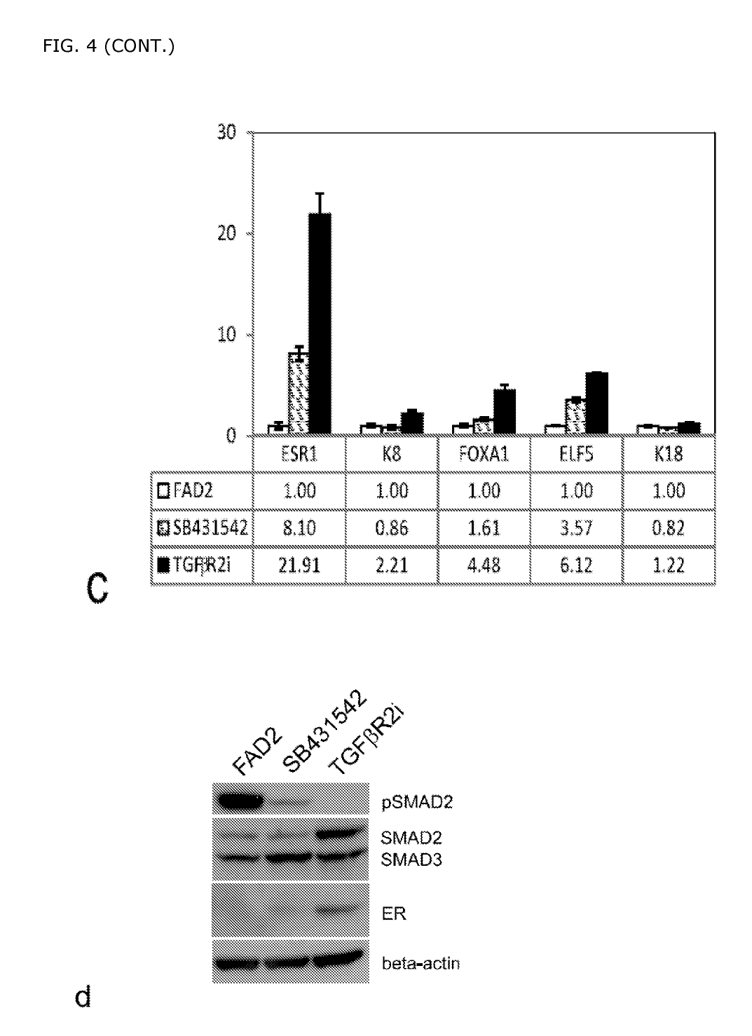

We noted that earlier in vivo studies had implicated TGFβ signaling in the restraint of ER positive mammary epithelial cells. Here we therefore examined three small inhibitor molecules of TGFβsignalling, RepSox, SB431542 and SD208 and combinations hereof for their ability to relieve a potentially negative regulation of ER^{pos} human breast epithelial cells growth in culture. We found that dual inhibition with SB431542 and RepSox, hereafter collectively termed TGFβR2i, recapitulated ER expression and stimulated ER^{pos} human breast epithelial cells colony formation in four out of four biopsies. Moreover, the cells maintained Ks20.8 reactivity (FIG. 4a). ER^{pos} cells from three different biopsies in low density primary cultures in TGFβR2i (4000 cells/cm²) exhibited a clonal capacity of 0.54%, 0.33%, and 0.43%, respectively. By comparison, control conditions resulted in small, mostly abortive ER^{neg} clones (FIG. 4b). In general, ER expression was particularly evident in the dense center of proliferating colonies or at confluency. Two different sources of SP1 antibody gave similar results (see Table 1). Moreover, staining was confirmed with the less sensitive ER antibody 1D5. The response to TGUR inhibitors was reproducibly observed in ten out of ten biopsies.

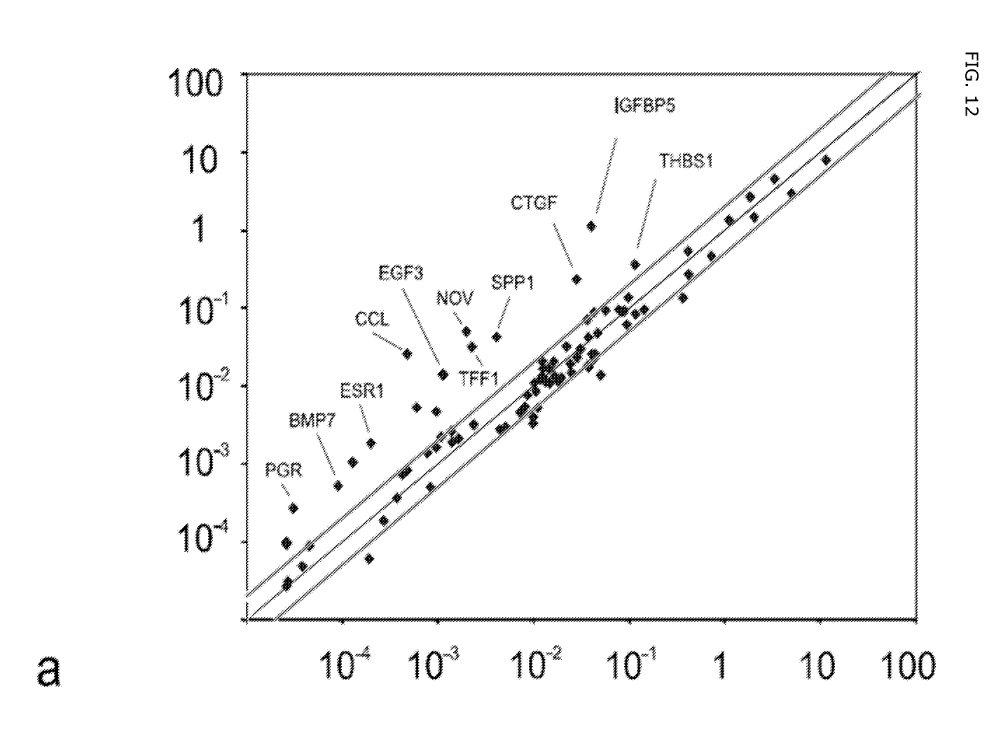

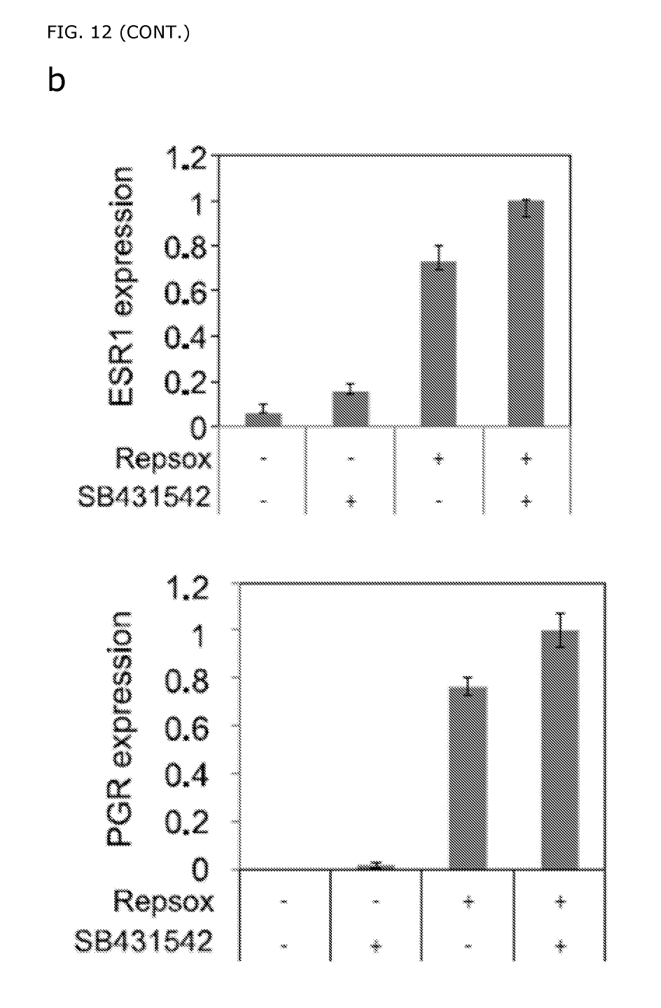

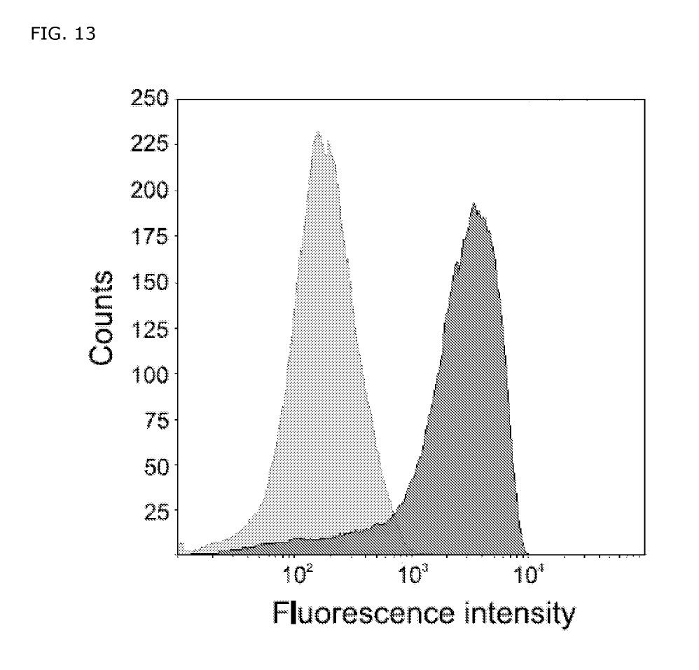

In addition to ER, TGFβR2i induced the expression of K8 and the luminal cell transcription factors forkhead box protein A1, FOXA1 and E74-like factor 5, ELF5 as revealed at the transcriptional level (FIG. 4c). This expression pattern was confirmed using another biopsy. TGFβR2i also upregulated transcription of a number of genes known to be downstream targets of ER or modulators of ER activity, such as TFF-1 and insulin growth factor binding protein 5 (IGFBP5) (FIG. 12a). Moreover, induction of ER protein expression to a significant degree apparently was specific to RepSox as the twice the concentration of SB431542 or replacement of RepSox with another ALK-5 kinase inhibitor, SD208, was insufficient to induce K8 and ER (data not shown). RepSox alone was not as effective in inducing ER or PR as in combination with SB431542 (FIG. 12b). Likewise, while SB431542 alone was capable of inducing increased expression of ER at the mRNA level (FIG. 4c), this translated to the protein level to a significant degree only in the presence of RepSox (FIG. 4d). These findings suggest that ER expression is controlled through specific inhibition of TGFβ signaling. The presence of TGFβR in HBEC as well as the inhibition of phosphorylated SMAD2 concurrent with inhibitor-induced ER expression supported this (FIG. 13 and FIG. 4d). Indeed, TGFβR2i culture was key to sustained ER protein expression as removal of TGFβR2i leads to complete loss of ER protein expression within 5 days (reduced to 10.7+/−0.5% ER^{pos} cells after 3 days and to 0% after 5 days without TGFβR2i as compared to 33.7+/−5.0% ER^{pos} cells in continuous TGFβR2i culture).

We next tested whether TGFβR2i would induce de novo ER expression in other subpopulations of luminal cells, for example in the much more frequent CD117^{high}-derived human breast epithelial cells. Consistent with the presumed progenitor status of CD117^{high} cells in the human breast, clones emerged from CD117^{high} cells that gained ER and stained positively for Ks20.8. This was, however, the only additional source we found of ER^{pos} human breast epithelial cells, since TGFβR2i failed to induce ER in basal cells, in breast fibroblasts or in the established normal cell breast lines, MCF10A or HMT3522 (data not shown). Thus, both from preexisting ER^{pos} human breast epithelial cells and from ER^{neg} progenitors, TGFβR2i readily provides growing colonies of ER^{pos} human breast epithelial cells.

TG93R2i Culture Allows for Long-Term Growth of ER^{pos} Human Breast Epithelial Cells.

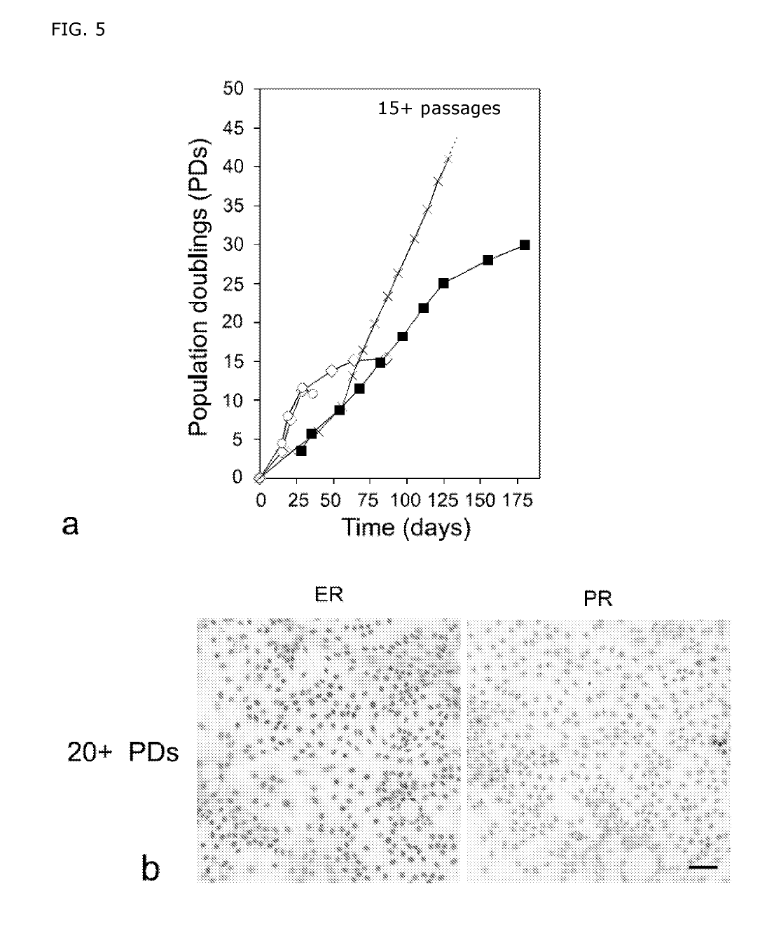

TGFβR2i appeared to support growth of ER^{pos} human breast epithelial cells also after passaging. To test this systematically, we plated CD166^{high}-derived cells at a density of 6400 cells/cm² in primary culture, and the cultures were subsequently passaged at a density of 4000 cells/cm². Upon passaging, ER expression was particularly evident in the dense center of proliferating colonies. Continuous proliferation under these conditions was maintained for up to six passages, corresponding to fifteen population doublings (FIG. 5a). In the absence of RepSox, however, the cells could not be expanded beyond fourth passage (FIG. 5a). The life span and ER expression level (ranging from 21-50% in second and third passage cultures) were somewhat biopsy dependent, and in general proliferation slowed between fourth and sixth passage, and at the same time, downregulation of ER expression was observed. While this narrowed the window of experimentation to up to third passage, the cells could easily and reproducibly be replaced with new cultures with similar luminal characteristics (Table 2).

TABLE 2
Frequency of estrogen receptor- and linage marker-positive
cells as a function of passage number in culture

| Passage # | Percent ER⁺ cells | Percent K8⁺ cells | Percent K19⁺ cells | Percent P63⁺ cells |
|---|---|---|---|---|
| 2 | 41/21/24/38/42 | 100/100/100/100 | 44/47/100/60 | 0*/5/0/0* |
| 3 | 47/22/50**/26 | 100/100/100/100 | 41/62/100/76 | 2/0/0/0 |
| 4 | 28/23 | 100/100 | 34/76 | 0/0 |
| 5 | 38^{•}/70^{†} | ND | 29^{†} | 0*^{†} |
| 6 | 42^{•} | ND | ND | ND |
| 7 | 30^{•} | ND | ND | ND |
| 8 | 34^{•} | 100^{•} | 100^{•} | 0^{•} |
| 9 | 28^{•}/87^{†} | 100^{•}/100***^{†} | 100^{•}/14^{†} | 0^{•}/0^{†} |
| 10 | 19^{•} | ND | ND | ND |

*Very few, small foci (<0.01%).
**+E2 in passage 1.
***Stained in passage 11.
^{•}= Cells with extended life span upon initial cultivation in BBMYAB and mouse feeders.
^{†}= hTERT/shp16 transduced cells.

Explanation to table 2: The percentage of ER- and lineage marker-positive cells evaluated after immunoperoxidase staining of cultures and counterstained with hematoxylin. Aside from staining for ER, the lineage markers included luminal keratin K8 (stained by M20 or TS1) and keratin K19, and myoepithelial P63. Each number refers to the frequency of stained cells among a total of 1000 cells in randomly selected fields in a culture representing one biopsy. Cells were counted by use of a 25× objective and a 10× ocular equipped with a grid. Note that cultures remain essentially ERpos and luminal-like. At the onset of senescence (starting at passage 4 in short-term cultures and in passage 10 in long-term cultures of cells with definite life span) there is a tendency for the ER staining to fade out.

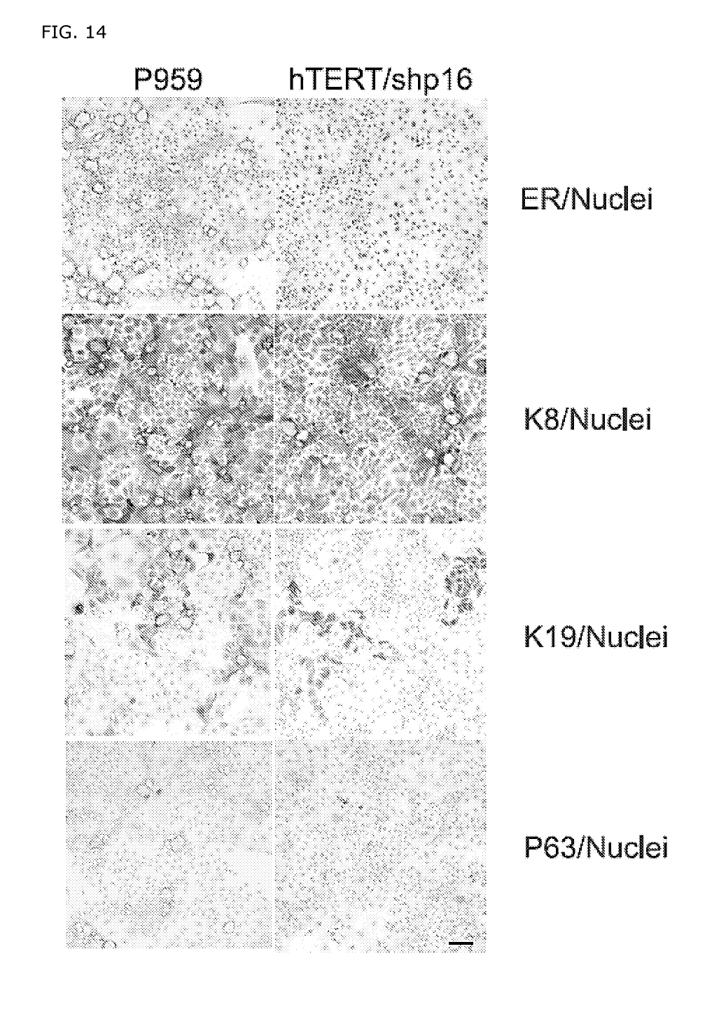

The life span of ER^{pos} human breast epithelial cells could be further extended by initial plating on 3T3 feeders, which leads to proliferation for more than ten passages, corresponding to more than 25 population doublings (FIG. 5a). We subsequently addressed whether TGFβR2i culture would allow for alternative approaches to extend the life span of normal breast-derived ER^{pos} cells. ER^{pos} HBEC were successfully transduced with pBABE-neo-hTERT and pLenti X2 hygro/shp16 constructs and have now been cultured for four months with weekly passages at 6000 cells/cm², exceeding 15 passages (FIG. 5a). Of note, these long-term cultured cells exhibited a phenotype essentially similar to cultures with definitive life span, including a relatively high level of ER expression (FIG. 14 and Table 2). Thus, while the proliferation of ER^{pos} cells with definite life span is somewhat slow as it took more than 100 days to generate more than 25 population doublings, the present protocol nevertheless allows a considerable expansion of the ER^{pos} cell population, which provides a relatively wide window of experimentation. Of note, until senescence the cells maintain their expression of ER, Ks20.8 reactivity as well mainly in densely packed colonies—expression of PR (FIG. 5b).

ER^{pos} Cells Respond to Estrogen.

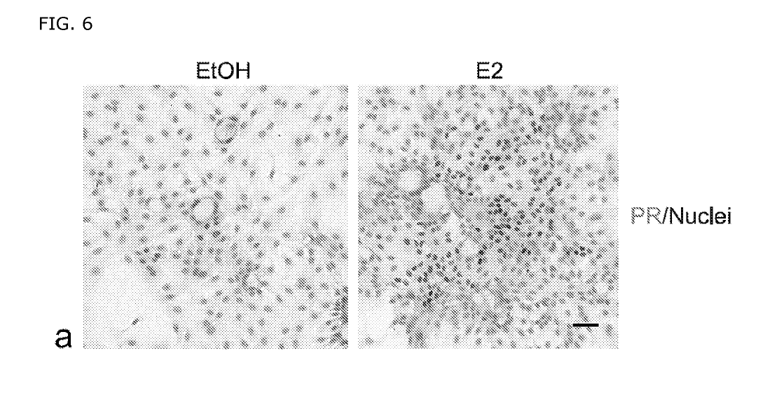

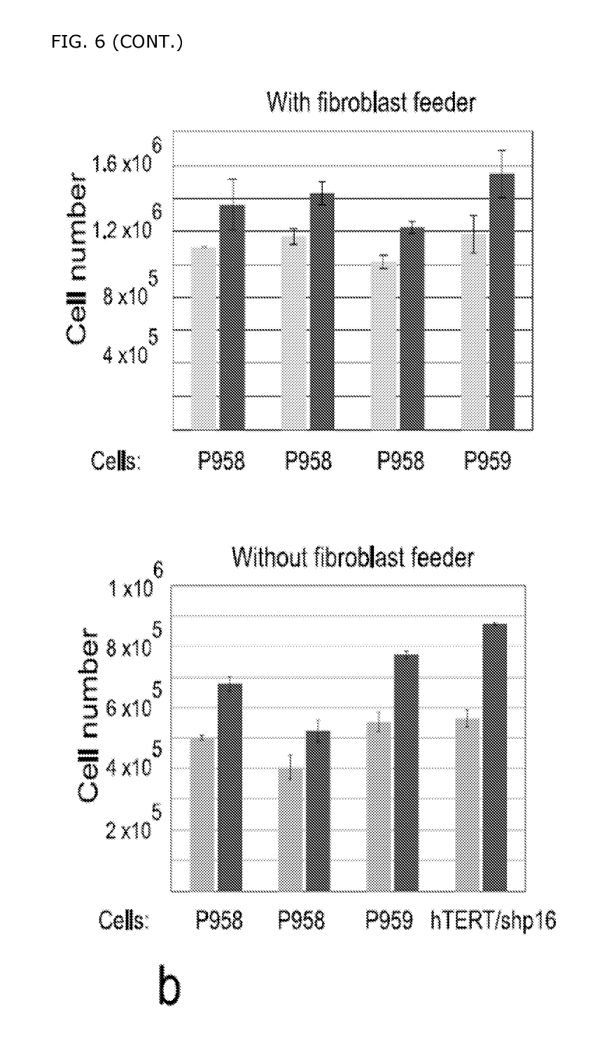

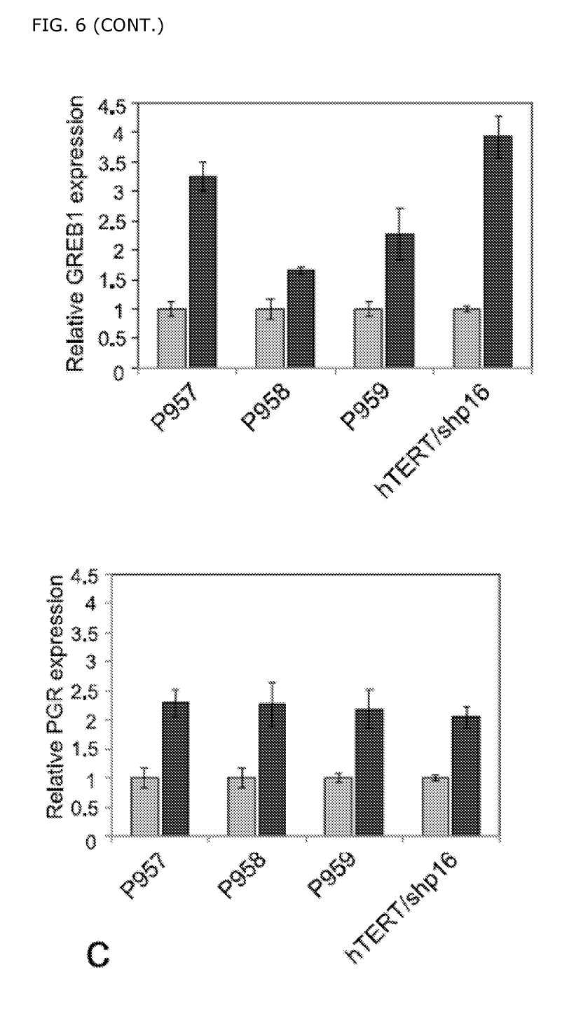

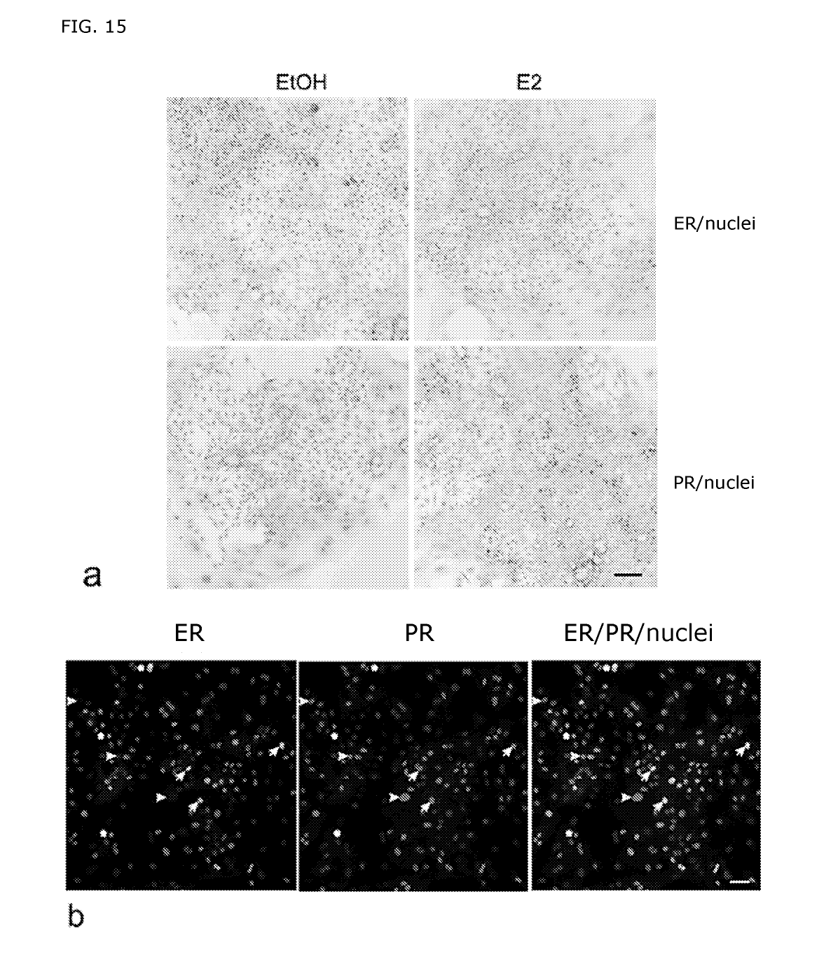

As an ultimate test for a physiologically relevant ER expression we decided to assess the effect of added cognate ligand, i. e. estrogen (β-estradiol). Accordingly, ER^{pos} cells were exposed to 10⁻⁸ M of β-estradiol or vehicle. The effect of estrogen was tested in the absence of epidermal growth factor (EGF) since the transcriptional activation of ER can be induced by EGF-induced MAPK activity. As a first indication of a functional ER, a higher focal expression of PR protein, a downstream target of ER signaling, was seen in the presence of estrogen (FIG. 6a). Early passage cultures were exposed to estrogen or vehicle, and after four days the cell number was quantified. As seen in FIG. 6b, significantly higher cell numbers were recorded in cultures with estrogen (FIG. 6b left panel). A similar proliferative response was observed in hTERT/shp16-transduced ER^{pos} cells (FIG. 6b, left panel). Long-term exposure to estrogen further augmented the estrogen response at the transcriptional level, as indicated by increased expression of PGR and GREB1 (FIG. 6c), as well as at the translational level (FIG. 15a) with a significant overlap in ER and PR protein expression (FIG. 15b). Thus, the isolated ER^{pos} cells retained their ability to respond to estrogen in a physiologically relevant manner.

As an alternative approach we plated ER^{pos} cells on fibroblast feeders which have been used previously to reveal an estrogenic response in mouse mammary cells. ER^{pos} cells were plated on confluent fibroblast feeders in TGFβR2i with or without estrogen. At day eight, there was a significant increase in cell number with estrogen as compared to the control (FIG. 6b right panel). In the presence of fibroblasts, ER^{pos} cells responded to estrogen irrespective of the presence of EGF (FIG. 6b). These data imply that the stromal microenvironment beyond the influence of TGF-β is important in the regulation of growth of ER^{pos} cells.

In conclusion, we have developed a method to isolate, track and long-term culture ER^{pos}-and estrogen-responsive cells from normal human breast.

Stable Luminal Phenotype.

CD166^{high}/CD117^{low} hTERT/shp16 cells cultured in FAD2+SB431542+RepSox (TGFbetaR2i) were sorted by FACS as CD146^{high} in passage 6 and 11 (FIG. 16). After culture until passage 22 the cells were sorted by FACS as EpCAM^{high}/CD146^{high}/CD117^{high} (P7 gate) and switched to FAD3+SB431542+RepSox. Subsequent cultures were split at up to 6000 cells/cm², and it was observed that the luminal phenotype was stable beyond passage 35.

B506 (hTERT/shp16, CD166^{high}/CD117^{low}) passage 6 cells were sorted with CD146 to yield F1221-P4 (FIG. 17A). Few K14+ foci were observed, mainly in large cells; few p63+ foci were observed, mainly in small cells. The majority of cells were ER+, K8+, K19+. Some cells were PR+. In general, there were more luminal cells in the F1221-P4 population than in the starting population B506.

F1221-P4 (hTERT/shp16, CD166hi/CD117lo/CD146hi) passage 11 cells were sorted with CD146 and termed F1223-P4 (FIG. 17B). Large cells still displayed some K14+ foci, small cells displayed p63+ foci, but in general the foci were fewer than in F1221-P4. Almost all cells were found to be ER+, K8+, K19+, some cells were PR+. In general, the phenotype of F1223-P4 was found more luminal than F1221-P4.

F1223-P4 (CD166hi/CD117lo/CD1462xhi) passage 14 cells were sorted with CD146 and termed F1224-P4 (FIG. 17C). The gate was reduced at the high end of FCS-A to remove large K14+ cells. The cells were all ER+, K8+, K19+, p63−. Some cells were PR+, and few were K14+. The luminal phenotype of F1224-P4 is more pronounced than F1223-P4 (now passage 23). The cell population was found to remain clean (no proliferating p63+, no proliferating K14+ cells).

### REFERENCES

Briand, P., Petersen, O. W. & van Deurs, B. A new diploid nontumorigenic human breast epithelial cell line isolated and propagated in chemically defined medium. In Vitro Cell Dev Biol 23, 181-188 (1987).

Clarke, R. B., Howell, A., Potten, C. S. & Anderson, E. Dissociation between steroid receptor expression and cell proliferation in the human breast. Cancer Res 57, 4987-4991 (1997).

Counter, C. M., et al. Dissociation among in vitro telomerase activity, telomere maintenance, and cellular immortalization. Proc Natl Acad Sci U S A 95, 14723-14728 (1998).

Garbe, J. C., et al. Molecular distinctions between stasis and telomere attrition senescence barriers shown by long-term culture of normal human mammary epithelial cells. Cancer Res 69, 7557-7568 (2009).

Gellibert, F., et al. Identification of 1,5-naphthyridine derivatives as a novel series of potent and selective TGF-b type I receptor inhibitors. J. Med. Chem. 47, 4494-4506 (2004).

Hayflick (1973) Tissue Culture Methods and Applications, P. F. Kruse, Jr. and M. K. Patterson, Jr. eds., p. 220 (Academic Press, New York).

Ichida, J. K., et al. A small-molecule inhibitor of TGF-b signaling replaces Sox2 in reprogramming by inducing Nanog. Cell Stem Cell 5, 491-503 (2009).

Ince, T. A., et al. Transformation of different human breast epithelial cell types leads to distinct tumor phenotypes. Cancer Cell 12, 160-170 (2007).

Kiyono, T., et al. Both Rb/p16lNK4a inactivation and telomerase activity are required to immortalize human epithelial cells. Nature 396, 84-88 (1998).

Liu, X., et al. ROCK inhibitor and feeder cells induce the conditional reprogramming of epithelial cells. Am J Pathol 180, 599-607 (2012).

Livak, K. J. & Schmittgen, T. D. Analysis of relative gene expression data using real-time quantitative PCR and the 2-2^{-DD}Ct method. Methods 25, 402-408 (2001).

Lundholt, B. K., Briand, P. & Lykkesfeldt, A. E. Growth inhibition and growth stimulation by estradiol of estrogen receptor transfected human breast epithelial cell lines involve different pathways. Breast Cancer Res. Treat. 67, 199-214 (2001).

Moll, R., Löwe, A., Laufer, J. & Franke, W. W. Cytokeratin 20 in human carcinomas. A new histodiagnostic marker detected by monoclonal antibodies. Am J Pathol 140, 427-447 (1992).

Pasic, L., et al. Sustained activation of the HER1-ERK1/2-RSK signaling pathway controls myoepithelial cell fate in human mammary tissue. Genes & Dev 25, 1641-1653 (2011).

Petersen, O. W. & van Deurs, B. Growth factor control of myoepithelial-cell differentiation in cultures of human mammary gland. Differentiation 39, 197-215 (1988).

Rønnov-Jessen, L. & Petersen, O. W. Induction of a-smooth muscle actin by transforming growth factor-b1 in quiescent human breast gland fibroblasts. Implications for myofibroblast generation in breast neoplasia. Lab. Invest. 68, 696-707 (1993).

Ronnov-Jessen, L., Celis, J. E., van Deurs, B. & Petersen, O. W. A fibroblast-associated antigen: Characterization in fibroblasts and immunoreactivity in smooth muscle differentiated stromal cells. J Histochem Cytochem 40, 475-486 (1992).

Shoker, B. S., et al. Estrogen receptor-positive proliferating cells in the normal and precancerous breast. Am J Pathol 155, 1811-1815 (1999).

Soule, H. D., et al. Isolation and characterization of a spontaneously immortalized human breast epithelial cell line, MCF-10. Cancer Res 50, 6075-6086 (1990).

Tan, D. W. M., et al. Single-cell gene expression profiling reveals funcional heterogeneity of undifferentiated human epidermal cells. Development 140, 1433-1444 (2013).

Zajchowski, D. A., Sager, R. & Webster, L. Estrogen inhibits the growth of estrogen receptor-negative, but not estrogen receptor-positive, human mammary epithelial cells expressing a recombinant estrogen receptor. Cancer Res 53, 5004-5011 (1993).

1. An immortalized estrogen receptor positive cell line, wherein said cell line has a CD326^{high}/CD271^{low} phenotype.

2. The cell line according to any one of the preceding claims, wherein said cell line further has a CD166^{high}/CD117^{low} phenotype.

3. The cell line according to any one of the preceding claims, wherein said cell line further has a Ks20.8^{high} phenotype.

4. The cell line according to any one of the preceding claims, wherein said cell line is capable of responding to estrogen.

5. The cell line according to any one of the preceding claims, wherein said cell line has been immortalized by genetic modification and/or transfection.

6. The cell line according to any one of the preceding claims, wherein said cell line has been immortalized by insertion of and/or transfection with of a telomerase reverse transcriptase such as a human telomerase reverse transcriptase (hTERT) gene and/or a shRNA P16 (shp16) gene.

7. The cell line according to any one of the preceding claims, wherein said cell line is derived from a primary breast epithelial progenitor cell.

8. The cell line according to any one of the preceding claims, wherein said cell line is derived from a primary breast epithelial luminal progenitor cell.

9. The cell line according to any one of the preceding claims, wherein said cell line is derived from a cell strain as defined in any one of the claims 21-22 and/or cell strain with extended lifespan as defined in any one of claims 17-20.

10. The cell line according to any one of the preceding claims, wherein said cell line is derived from a primary cell isolated as defined in any one of the claims 14-16.

11. The cell line according to any one of the preceding claims, wherein said cell line is cultured in a culture medium A according to any one of the claims 25-37.

12. The cell line according to any one of the preceding claims, wherein said cell line remains estrogen receptor positive for at least 25 population doublings.

13. The cell line according to any one of the preceding claims, wherein said cell line is cultured in Primaria flasks.

14. A method of isolating a primary breast epithelial cell which is capable of establishing the estrogen receptor positive cell line of claims 1-13 and/or the estrogen receptor positive cell strain with extended lifespan of claims 17-20 and/or the cell strain of claims 21-22, said method comprising the steps of:
a. providing a sample of breast epithelium cells; and
b. isolating a primary cell with a CD326^{high}/CD271^{low} phenotype thereby isolating a primary breast epithelial cell capable of yielding a cell line according to any one of the preceding claims and/or cell strain with extended lifespan according to any one of the claims 17-20 and/or the cell strain according to any one of the claims 21-22.

15. The method according to claim 14, wherein said isolated cell further has a CD166^{high}/CD117^{low} and/or CD166^{low}/CD117^{high} phenotype.

16. The method according to any one of claims 14-15, wherein said cell further has a Ks20.8^{high} and/or Ks20.8^{low} phenotype.

17. A method of generating an estrogen receptor positive cell strain with extended lifespan from an isolated breast epithelial cell, the method comprising the steps of:
a. isolating a breast epithelial cell according to any one of the preceding claims; and
b. culturing said isolated cell in presence of at least one feeder cell in culture medium B according to claims 38-39,
c. isolating a cell with a CD326^{high}/CD271^{high} phenotype and/or a CD326^{high}/CD271^{low} phenotype, and
d. culturing said isolated cell of c in culture medium A according to claims 25-37,
wherein said isolated cell generates an estrogen receptor positive cell strain with extended lifespan capable of responding to estrogen.

18. The method according to claim 17, wherein said feeder cell comprises a fibroblast cell.

19. The method according to any one of claims 17-18, wherein said feeder cell comprises NIH-3T3 murine fibroblast cells.

20. The method according to any one of claims 17-19, wherein said cell strain with extended lifespan remains estrogen receptor positive for at least 10 population doublings.

21. A method of generating an estrogen receptor positive cell strain from an isolated breast epithelial cell, the method comprising the steps of:
a. isolating a breast epithelial cell according to any one of the preceding claims; and
b. culturing said isolated cell in culture medium A according to any one of the claims 25 to 37,
wherein said isolated cell generates an estrogen receptor positive cell strain capable of responding to estrogen.

22. The cell strain according to claim 21, wherein said cell strain is cultured in Primaria flasks.

23. A method of distinguishing an estrogen receptor positive cell and an estrogen receptor negative cell based on cell surface proteins, said method comprising the steps of:
a. providing a cell; and
b. determining if said cell has a Ks20.8^{high} or Ks20.8^{low} phenotype;
wherein a cell with a Ks20.8^{high} phenotype is estrogen receptor positive and a cell with a Ks20.8^{low} phenotype is estrogen receptor negative.

24. The method according to claim 22, wherein said estrogen receptor positive cell further has a CD326^{high}/CD271^{low} phenotype and/or a CD166^{high}/CD117^{low} phenotype.

25. A cell culture medium A for inducing and/or maintaining an estrogen receptor positive phenotype in a breast epithelial cell, cell strain with extended lifespan and/or a cell line according to any one of the preceding claims, wherein the culture medium comprises an inhibitor of a TGF-β type I receptor.

26. The culture medium A according to claim 25, wherein said inhibitor of a TGF-β type I receptor comprises an inhibitor of a TGF-β type I receptor activin receptor-like kinase and/or an inhibitor of a TGF-β type I receptor activin receptor-like kinase autophosphorylation.

27. The culture medium A according to any one of claims 25-26, wherein said inhibitor of a TGF-β type I receptor comprises one or more compounds selected from the group comprising of TGF-f3R2i, SB431542, RepSox, and SD208.

28. The culture medium A according to any one of claims 25-27, further comprising a Rho-associated coiled coil forming protein serine/threonine kinase inhibitor.

29. The culture medium A according to any one of claims 25-28, wherein said Rho-associated coiled coil forming protein serine/threonine kinase inhibitor is selected from the group comprising Y-27632 and related compounds.

30. The culture medium A according to any one of claims 25-29, wherein the culture medium comprises:
a mixture of Dulbecco's modified Eagle's medium (DMEM, high glucose, no calcium, Life Technologies):Ham's F12 Nutrient Mixture (F12, Life Technologies) in the ratio around 3:1 v/v; and
around 0.5 μg/ml hydrocortisone; and
around 5 μg/ml insulin; and
around 10 ng/ml cholera toxin; and
around 10 ng/ml epidermal growth factor and/or around 5 nM amphiregulin; and
around 1.8×10⁻⁴ M adenine; and
around 10 μM Y-27632; and
around 5% fetal bovine serum modified as described in the present invention; and
around 10 μM SB431542; and
around 50 μM RepSox; and
around 2 mM L-glutamine.

31. The culture medium A according to any one of claims 25-30, wherein the culture medium is devoid of hydrocortisone and/or of cholera toxin.

32. The culture medium A according to any one of claims 25-31, wherein the culture medium comprises:
a mixture of Dulbecco's modified Eagle's medium (DMEM, high glucose, no calcium, Life Technologies):Ham's F12 Nutrient Mixture (F12, Life Technologies) in the ratio around 3:1 v/v; and
around 5 μg/ml insulin; and
around 10 ng/ml epidermal growth factor and/or around 5 nM amphiregulin; and
around 1.8×10⁻⁴ M adenine; and
around 10 μM Y-27632; and
around 5% fetal bovine serum modified as described in the present invention; and
around 10 μM SB431542; and
around 50 μM RepSox; and
around 2 mM L-glutamine;
wherein said medium is devoid of hydrocortisone and/or of cholera toxin.

33. The culture medium A according to any one of claims 25-32, wherein the culture medium comprises:
a mixture of Dulbecco's modified Eagle's medium (DMEM, high glucose, no calcium, Life Technologies):Ham's F12 Nutrient Mixture (F12, Life Technologies) in the ratio between 1:100 and 100:1 v/v; and
0.005-50 μg/ml hydrocortisone; and
0.05-500 μg/ml insulin; and
0.1-1000 ng/ml cholera toxin; and
0.1-1000 ng/ml epidermal growth factor and/or around 5 nM amphiregulin; and
0.02×10⁻⁴-200×10⁻⁴ M adenine; and
10 μM Y-27632; and
0.05%-500% fetal bovine serum modified as described in the present invention; and
0.1-1000 μM SB431542; and
0.25-250 μM or 0.50-500 μM RepSox; and
0.02-200 mM L-glutamine.

34. The culture medium A according to any one of claims 25-33, wherein the culture medium comprises:
a mixture of Dulbecco's modified Eagle's medium (DMEM, high glucose, no calcium, Life Technologies):Ham's F12 Nutrient Mixture (F12, Life Technologies) in the ratio 3:1 v/v; and
0.5 μg/ml hydrocortisone; and
5 μg/ml insulin; and
10 ng/ml cholera toxin; and
10 ng/ml epidermal growth factor and/or around 5 nM amphiregulin; and
1.8×10⁻⁴ M adenine; and
10 μM Y-27632; and
5% fetal bovine serum modified as described in the present invention; and
10 μM SB431542; and
25 μM or 50 μM RepSox; and
2 mM L-glutamine.

35. The culture medium A according to any one of claims 25-34, wherein the culture medium comprises:
a mixture of Dulbecco's modified Eagle's medium (DMEM, high glucose, no calcium, Life Technologies):Ham's F12 Nutrient Mixture (F12, Life Technologies) in the ratio around 3:1 v/v; and
around 5 μg/ml insulin; and
around 10 ng/ml epidermal growth factor and/or around 5 nM amphiregulin; and
around 1.8×10⁻⁴ M adenine; and
around 10 μM Y-27632; and
around 5% fetal bovine serum modified as described in the present invention; and
around 10 μM SB431542; and
around 25 μM or 50 μM RepSox; and
around 2 mM L-glutamine.

36. The culture medium A according to any one of claims 25-35, wherein the culture medium comprises:
a mixture of Dulbecco's modified Eagle's medium (DMEM, high glucose, no calcium, Life Technologies):Ham's F12 Nutrient Mixture (F12, Life Technologies) in the ratio between 1:100 and 100:1 v/v; and
0.05-500 μg/ml insulin; and
0.1-1000 ng/ml epidermal growth factor and/or around 5 nM amphiregulin; and
0.02×10⁻⁴-200×10⁻⁴ M adenine; and
10 μM Y-27632; and
0.05%-500% fetal bovine serum modified as described in the present invention; and
0.1-1000 μM SB431542; and
0.25-250 μM or 0.50-500 μM RepSox; and
0.02-200 mM L-glutamine.

37. The culture medium A according to any one of claims 25-36, wherein the culture medium comprises:
a mixture of Dulbecco's modified Eagle's medium (DMEM, high glucose, no calcium, Life Technologies):Ham's F12 Nutrient Mixture (F12, Life Technologies) in the ratio 3:1 v/v; and
5 μg/ml insulin; and
10 ng/ml epidermal growth factor and/or around 5 nM amphiregulin; and
1.8×10⁻⁴ M adenine; and
10 μM Y-27632; and
5% fetal bovine serum modified as described in the present invention; and
10 μM SB431542; and
25 μM or 50 μM RepSox; and
2 mM L-glutamine.

38. A culture medium B for generating a CD326^{high}/CD271^{high} breast epithelial cell, wherein the culture medium comprises a Rho-associated coiled coil forming protein serine/threonine kinase inhibitor, adenine and/or a serum replacement, such as B27.

39. The culture medium B according to claim 30, further comprising BBM medium without HEPES.

40. A method for producing a tumorigenic cell from an estrogen receptor positive cell, cell strain with extended lifespan and/or cell line and/or cell strain, the method comprising the step of:
a. providing estrogen receptor positive cell, cell strain with extended lifespan and/or cell line and/or cell strain according to any one of the preceding claims; and
b. contacting said cell to a tumorigenic agent which transforms said cell, cell strain with extended lifespan and/or cell line and/or cell strain into tumorigenic cells capable of forming a tumor in vitro or in vivo.

41. A tumorigenic cell, cell strain with extended lifespan and/or cell line and/or cell strain produced according to any one of the preceding claims.

42. An in vitro method for identifying an agent which reduces proliferation and/or survival of a tumorigenic cell, cell strain with extended lifespan and/or cell and/or cell strain according to any one of the proceeding claims, comprising the steps of:
a. contacting a tumorigenic cell, cell strain with extended lifespan and/or cell line and/or cell strain according to any one of the preceding claims with a candidate agent;
b. assessing the ability of said candidate agent for its ability to reduce proliferation and/or survival of said tumorigenic cell, cell strain with extended lifespan and/or cell line and/or cell strain;
c. determining the extent to which proliferation of the tumorigenic cell, cell strain with extended lifespan and/or cell line and/or cell strain occurs in the presence of the candidate agent; and
d. comparing the extent determined with the extent to which proliferation of the tumorigenic cell, cell strain with extended lifespan and/or cell line and/or cell strain occurs under the same conditions, but in absence of the candidate agent,
wherein if the proliferation and/or survival occurs to a lesser extent in the presence of the candidate agent than in its absence, the candidate agent is an agent which reduces proliferation and/or survival of said tumorigenic cell, cell strain with extended lifespan and/or cell line and/or cell strain.

43. A method of identifying a gene the expression of which in a tumorigenic cell, cell strain with extended lifespan and/or cell line and/or cell strain is related to or involved in metastasis of such cell in vivo, the method comprising the steps of:
a. introducing a candidate gene into a tumorigenic cell, cell strain with extended lifespan and/or cell line and/or cell strain according to any one of the preceding claims, thereby producing a modified tumorigenic cell, cell strain with extended lifespan and/or cell line;
b. introducing the modified tumorigenic cell, cell strain with extended lifespan and/or cell line and/or cell strain into a test animal;
c. maintaining said test animal under conditions appropriate for metastasis to occur; and
d. determining whether metastasis of the modified tumorigenic cell, cell strain with extended lifespan and/or cell line and/or cell strain occur,
wherein if metastasis occurs, the candidate gene is a gene the expression of which in a tumorigenic cell, cell strain with extended lifespan and/or cell line and/or cell strain is related to or involved in metastasis of such cells in vivo.

44. A method of identifying a gene product which is upregulated or downregulated in a tumorigenic cell, but not in a normal cell of the same type, the method comprising the steps of:
a. analyzing the tumorigenic cell, cell strain with extended lifespan and/or cell line and/or cell strain according to any one of the preceding claims;
b. analyzing a normal cell from which said tumorigenic cell, cell strain with extended lifespan and/or cell line and/or cell strain is derived; and
c. comparing the gene products produced in step (a) with step (b), whereby a gene product which is upregulated or downregulated is identified.

45. A method of culturing cancer cells, tumorigenic cells and/or tumors from normal and/or luminal epithelial cells, wherein said normal and/or luminal epithelial cells are grown in a cholera toxin free medium comprising FAD2, TGFβR inhibitor, with or without estrogen or in BBMYAB on fibroblast feeders with or without estrogen.

46. The method of claim 45, wherein the medium further is devoid of hydrocortisone.

47. The method of any one of claims 45-46, wherein the medium is the medium of any one of claim 25-37 or 38-39.
